(19) United States
(12) Patent Application Publication
(10) Pub. No.: US 2024/0358910 A1
(43) Pub. Date: Oct. 31, 2024

(54) A SYSTEM FOR TREATING A WOUND

(71) Applicant: AROA BIOSURGERY LIMITED, Auckland (NZ)

(72) Inventors: Brian Roderick Ward, Pukekohe (NZ); Issac Tristram Tane MASON, Auckland (NZ); Dorrin ASEFI, Auckland (NZ); Hamish Joshua Rose, Auckland (NZ); Sean James GORMAN, Auckland (NZ); Henry David CHITTOCK, Auckland (NZ)

(21) Appl. No.: 18/038,353

(22) PCT Filed: Nov. 24, 2021
(86) PCT No.: PCT/NZ2021/050205

§ 371 Date: May 23, 2023

Related U.S. Application Data
(60) Provisional application No. 63/117,995, filed on Nov. 24, 2020.

(51) Int. Cl. A61M 1/00

(52) U.S. Cl. CPC A61M 1/96; A61M 1/92; A61M 2205/3351; A61M 2205/3355

(57) ABSTRACT

A system for treating a wound, having a wound treatment device, a fluid input upstream side of the device, a fluid output downstream of the device; an air inlet valve upstream of the fluid output; an actuator to drive the air inlet valve between open and closed positions; a pump downstream of the fluid input; a motor to drive the pump to provide a negative pressure to the device; and a controller in configured to operate the air inlet valve and the pump. The controller is configured to (i) open the air inlet valve and operate the pump to maintain a first vacuum pressure at the wound treatment device and introduce air into the device, and (ii) close the air inlet valve and operate the pump to maintain a second vacuum pressure at the wound treatment device and remove air and fluid from the device.

### CROSS-REFERENCE TO RELATED APPLICATIONS

This application is a 35 U.S.C. § 371 national stage filing of International Application No. PCT/NZ2021/050205, filed Nov. 24, 2021, which claims priority to U.S. provisional application No. 63/117,995, filed Nov. 24, 2020. The contents of the aforementioned applications are hereby incorporated by reference in their entirety.

### FIELD OF THE INVENTION

This invention relates to a system for treating a wound, and in particular a system for removing fluid from a wound, and a system for supplying fluid to and removing fluid from a wound, and related components for such systems.

### BACKGROUND

The technique of applying negative pressure to manage wound exudate and accelerate the healing of various wounds has been utilised for some time. Applying negative pressure to an external wound to affect a therapeutic benefit is commonly referred to as negative pressure wound therapy or NPWT. This therapy can accelerate the formation of granulation tissue in open external wounds such as diabetic foot ulcers, dehisced surgical wounds, and various acute and chronic wounds to support healing by secondary and tertiary intention. A variety of special topical dressing systems are applied to these wounds to port negative pressure from a negative pressure source to a wound (FIG. 1). Internal closed surgical wounds have also been treated by applying suction (a vacuum) to a surgical site via an internally placed drain or device to remove wound fluids following surgery.

The applied negative pressure reduces the free volume of the wound treatment space to draw wound exudate towards the source of negative pressure, where the exudate is discharged into a reservoir or collection dressing that typically lies between the wound and the source of negative pressure.

Negative pressure is provided to a wound treatment space by a vacuum unit or vacuum device. Vacuum devices for wound treatment face a number of design challenges. The vacuum devices are either disposable or reusable, and ideally need to be readily portable, therefore, components should be lightweight, energy efficient and preferably inexpensive.

Existing systems can be susceptible to becoming blocked by coagulated blood, fibrin, adipose tissue, lose tissue debris and wound exudate. The mechanical vacuum pumps for use in negative pressure treatment systems can also be susceptible to blockage, particularly where the vacuum systems are small in size. In addition, vacuum systems are typically remotely located from the wound where the accurate measurement and regulation of pressure directly at the target site can be difficult to achieve, due to the potential for blockages to occur within the mechanical vacuum pump assembly or elsewhere in the system, and the changing volume within the fluid collection reservoirs.

Some prior art systems comprise a pressure relief valve to control the negative pressure at a wound, for example as described in US2007/0179460. A pressure relief valve operates to introduce air to the system to prevent the applied vacuum pressure increasing above an upper threshold pressure, or to return the system to ambient pressure. Such systems can result in a loss of negative pressure at the wound treatment site and may negatively impact the wound healing process.

Once a system has reached a desired negative pressure the system typically remains sealed or closed from the ambient environment, with the only input being exudate produced at the wound site. A system can reach an equilibrium state, resulting in the wound fluid retained within system becoming static or stagnant even when additional exudate is produced at the wound site. This static or stagnant fluid can further exacerbate the coagulation of blood, settling of tissue debris and the formation of fibrin that can lead to an increased risk of blockage and failure in the application of negative pressure at the wound. Furthermore, the stagnation of excess wound exudate can increase the risk of infection, oedema and may also lead to biofilm formation and subsequent stalled healing.

A further difficulty with administering negative pressure treatment is there is often a height difference at the wound site, for example when the patient is upright or in a standing position. A height differential at the wound can result in preferential flow of fluid from upper portions of the wound, with fluid remaining static in a lowermost portion of the wound.

In addition to applying negative pressure to a wound for therapeutic pressure treatment, the ability to instil fluid to and across the wound site can be helpful to administer wound cleansing fluids, saline, pain medications, cell suspensions, treatment solutions and other liquid medications to supress bacteria or to flush a wound.

Existing vacuum devices that can also instil treatment fluids suffer the same design limitations as the standard non-administering variants. They are typically large in size due to the integrated rigid waste collection containers that are positioned between the vacuum pump and the wound, and/or require a large amount of energy to power the pumping components within the device which can add to the size and complexity of the system.

The treatment fluid is typically delivered to the wound via a fluid supply conduit which is subjected to positive pressure to ensures complete saturation of the wound site, resulting in the wound site remaining at an ambient or positive pressure level. These systems then subsequently apply a vacuum to the wound site to draw the treatment fluid and exudate away from the wound site via the reduction of the free volume of the treatment space in response to the applied negative pressure, which is subsequently collected in the attached reservoir or collection container. The positive pressure applied to the wound treatment site can have unintended consequences such as inducing a leak from the wound dressing, typically between the peri-wound and a cover dressing. The loss of vacuum pressure can also cause various elements of the special topical wound dressings to move such as the foam porting layer/wound interface or the fluid supply and exudate fluid conduits, which may lead to a short circuit in the fluid flow path through the wound. This short circuiting may result in zero treatment fluid delivery to the wound.

It is an object of the present invention to address one or more of the abovementioned disadvantages and/or to at least provide the public with a useful alternative.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF THE INVENTION

In a first aspect, the present invention provides a system for treating a wound comprising:
a fluid input and a fluid output for connection to a wound treatment device located at the wound, the fluid input adapted to be fluidly connected to an upstream side of the wound treatment device and the fluid output adapted to be fluidly connected to a downstream side of the wound treatment device;
an air inlet valve upstream of the fluid output;
an actuator to drive the air inlet valve between an open position and a closed position;
a pump downstream of the fluid input;
a motor to drive the pump to provide a negative pressure to the wound treatment device; and
a controller in communication with the actuator and the motor to operate the air inlet valve and the pump; wherein the controller is configured to:
   i). open the air inlet valve and operate the pump to maintain a first vacuum pressure at the wound treatment device and introduce air into the wound treatment device;
   ii). close the air inlet valve and operate the pump to maintain a second vacuum pressure at the wound treatment device and remove air and fluid from the wound treatment device;
   wherein the first vacuum pressure is less than or equal to the second vacuum pressure.

In a second aspect, the present invention provides a system for treating a wound comprising:
a fluid input and a fluid output for connection to a wound treatment device located at the wound, the fluid input adapted to be fluidly connected to an upstream side of the wound treatment device and the fluid output to be fluidly connected to a downstream side of the wound treatment device;
an air inlet valve upstream of the fluid output and an actuator to drive the air inlet valve between an open position and a closed position;
a treatment fluid inlet upstream of the fluid outlet to connect a supply of treatment fluid;
a treatment fluid valve between the treatment fluid inlet and the fluid outlet and an actuator to drive the fluid inlet valve between an open position and a closed position;
a pump downstream of the fluid input;
a motor to drive the pump to provide a negative pressure to the wound treatment device; and
a controller in communication with the air inlet valve actuator, the fluid valve actuator and the motor to operate the air inlet valve, the fluid valve and the pump; wherein the controller is configured to:
   i). open the air inlet valve and operate the pump to maintain a first vacuum pressure at the wound treatment device and introduce air into the wound treatment device;
   ii). close the air inlet valve and operate the pump to maintain a second vacuum pressure at the wound treatment device and remove air and fluid from the wound treatment device, wherein the first vacuum pressure is less than or equal to the second vacuum pressure; and
   in a fluid supply state and with the air inlet valve closed:
      iii). open the fluid inlet valve and operate the pump to maintain a vacuum pressure at the wound treatment device and introduce treatment fluid into the wound treatment device;
      iv). close the fluid inlet valve and operate the pump to maintain a vacuum pressure at the wound treatment device and remove fluid from the wound treatment device.

The first or second aspects of the invention may include any one or more features described in relation to the third, fourth and fifth aspects of the invention.

In a third aspect, the present invention provides a pump for applying negative pressure to a wound via a wound treatment device, the pump comprising:
a drive mechanism;
at least one flexible chamber, the drive mechanism configured to drive the chamber to compress and expand the chamber;
a pair of one-way valves in fluid communication with the chamber, the pair of one-way valves comprising an inlet valve for fluid flow into the chamber, and an outlet valve for fluid flow out of the chamber;
a pump inlet in fluid communication with the at least one inlet valve; and
a pump outlet in fluid communication with the at least one outlet valve;
wherein compression of the chamber causes fluid flow from the chamber, through the outlet valve and the pump outlet, and subsequent expansion of the chamber draws fluid from the pump inlet through the inlet valve and into the chamber; and
wherein the one-way inlet and outlet valves each presents a single orifice only in a fluid flow path through the pump from the pump inlet to the pump outlet via the inlet valve, chamber and outlet valve to enable the passage of fluid and tissue debris through the valves when open.

In a fourth aspect, the present invention provides a wound treatment device for applying negative pressure to an external wound, the device comprising:
a porting component to be received in an external wound cavity and substantially fill a treatment space of the wound;
a cover layer to cover the wound;
a fluid supply conduit, the fluid supply conduit having one or more supply conduit outlets;
a fluid removal conduit, the fluid removal conduit having one or more removal conduit inlets;
wherein the supply and removal conduits are placed in the treatment space with the removal conduit inlet(s) and the supply conduit outlet(s) in fluid communication with the porting component and with the outlet(s) spaced from the inlet(s) so that fluid flow from the outlet(s) to the inlet(s) is through a substantial portion of the porting component and treatment space.

In a fifth aspect, the present invention provides a portable vacuum unit for a wound treatment system for providing negative pressure treatment to a wound, the vacuum unit comprising:
an air inlet valve;
an actuator to drive the air inlet valve between an open position and a closed position;
a pump comprising a pump inlet and a pump outlet;
a motor to drive the pump; and
a controller in communication with the actuator and the motor to operate the air inlet valve and the pump to apply negative pressure treatment to a wound, and
an interface manifold comprising:
   a first fluid flow path with a first inlet and first outlet, the first inlet connected to the air inlet valve and the first outlet providing a vacuum unit fluid outlet for connection to an upstream side of the treatment device, and
   a second fluid flow path with a second fluid inlet and a second fluid outlet, the second outlet connected to the pump inlet and the second inlet providing a vacuum unit fluid inlet for connecting to a downstream side of the treatment device,
an enclosure for housing the air inlet valve, actuator, pump, motor, controller, and interface manifold,
wherein the interface manifold is a separate assembly within the enclosure providing an interface between the air inlet valve and the upstream side of wound treatment device and an interface between the pump inlet and the downstream side of the wound treatment device.

Further features of the above aspects of the invention are set out in the appended claims.

### Definitions

In this specification and claims, unless the context indicates otherwise, the term ‘exudate’ is intended to mean any fluid removed from a wound site of a patient. Exudate may comprise exudate produced by the patient, and/or fluid applied to the wound site by the system, including air or treatment fluid such as saline or fluid providing medication etc, or via a surgical intervention that may have introduced or administered treatment fluids to the wound site via a separate route; such as injection.

In this specification and claims, unless the context indicates otherwise, the terms ‘fluid’ and ‘treatment fluid’ are intended to mean liquid fluids and liquid treatment fluids such as wound irrigation solutions. Thus, unless the context suggests otherwise, the terms ‘fluid’ and ‘liquid’ may be used interchangeably.

In this specification and claims, the terms ‘negative pressure’ and ‘vacuum pressure’ may be used interchangeable to mean a gauge pressure less than an ambient pressure and an absolute pressure less than atmospheric pressure, which can also be referred to as sub-atmospheric pressure or suction pressure. For example, a negative pressure or vacuum pressure of 100 mmHg is −100 mmHg gauge pressure or around 660 mmHg absolute pressure. The terms ‘high’, ‘increase’ or other similar terms when used in relation to negative or vacuum pressure are intended to mean higher or increasing negative pressure, for example a gauge pressure of −150 mmHg (610 mmHg absolute) may be described as being ‘higher’ than a gauge pressure of −100 mmHg (660 mmHg absolute). Similarly, in relation to the terms ‘low’, ‘decrease’ or other similar terms when used in relation to negative or vacuum pressure, a gauge pressure of −100 mmHg may be described as being ‘lower’ than a gauge pressure of −150 mmHg.

In this specification and claims, unless the context indicates otherwise, the term ‘NPT’ is intended to mean negative pressure treatment which relates to a system or apparatus that is configured to administer ‘negative pressure’ or ‘vacuum pressure’ to provide treatment to any internal or external wound. For the purposes of clarity, a system that is configured to administer negative pressure wound therapy to an external wound is considered a type of negative pressure treatment system, as is a system that is configured to administer suction to an internal closed surgical site.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE DRAWINGS

The present invention will now be described by way of example only and with reference to the accompanying drawings in which:

FIG. 1 is a schematic of a prior art negative pressure treatment system which is configured to treat an external wound;

FIG. 2 provides a high level schematic representation of a negative pressure treatment (NPT) system according to at least one embodiment described herein;

FIG. 3 illustrates the system of FIG. 2 applied to an external wound;

FIG. 4 illustrates the system of FIG. 2 applied to an internal wound;

FIG. 5(i) is a schematic representation of a vacuum unit of the system of FIG. 2. FIGS. 5(ii) and 5(iii) are a schematic section views of a dual lumen conduit taken through planes A and B of FIG. 5(i);

FIG. 6 is a schematic representation of the system of FIG. 2;

FIG. 7 is a further schematic representation of the system of FIG. 2 and is equivalent to the schematic of FIG. 6 but additionally illustrating additional components of the vacuum unit of the system;

FIG. 8 is a schematic representation of an alternative embodiment of an NPT system comprising features of the system of FIG. 2 and additionally including a treatment fluid reservoir;

FIG. 9 is a schematic representation of the system of FIG. 8;

FIG. 10 is a further schematic representation of the system of FIG. 8 and is equivalent to the schematic of FIG. 9 but additionally illustrating additional components of a vacuum unit of the system;

FIG. 11 is a schematic representation of a further alternative embodiment of an NPT system;

FIG. 12 is a further schematic representation equivalent to the schematic of FIG. 11 but additionally illustrating additional components of a vacuum unit of the system;

FIG. 13 provides two views of a pump for use in an NPT system such as those systems described herein;

FIG. 14 is a cross sectional view of the pump of FIG. 13;

FIG. 15 is an exploded view of the pump of FIG. 13;

FIG. 16 is another exploded view of the pump of FIG. 13;

FIG. 17 illustrates the pump of FIG. 13 together with an interface manifold removably attached to an inlet of the pump, corresponding with the system of FIGS. 11 and 12. The manifold can also be adapted for use with the system of FIGS. 9 and 10;

FIG. 18 illustrates the pump and interface manifold from FIG. 17 with the interface manifold separated from the pump;

FIG. 19 illustrates the interface manifold from FIGS. 17 and 18;

FIG. 20 provides two views of an interface manifold much the same as the interface manifold shown in FIGS. 17 to 19;

FIG. 21 is a cross sectional view of the manifold of FIG. 20 with the section through the manifold indicated in ends views presented in the Figure;

FIG. 22 is another cross sectional view of the manifold of FIG. 20 with the section through the manifold indicated in ends views presented in the Figure;

FIG. 23 is an exploded view of the manifold of FIG. 20;

FIG. 24 illustrates an alternative interface manifold corresponding with the system shown in FIGS. 6 and 7;

FIG. 25 is an exploded view of the manifold of FIG. 24;

FIG. 26 is a cross sectional view of the manifold of FIG. 24 with the section through the manifold indicated in ends views presented in the Figure;

FIG. 27 illustrates an alternative pump together with a motor for driving the pump;

FIG. 28 is an exploded view of the pump of FIG. 27;

FIG. 29 is an end view on a duck bill valve that may be incorporated in the pump assemblies described herein;

FIG. 30 is a sectional view through the duck bill valve of FIG. 29;

FIG. 31 shows a cross section of a dual lumen conduit;

FIG. 32 shows a cross section of an alternative dual lumen conduit;

FIG. 33 provides a front view and side views of a portable vacuum unit for the system of FIGS. 5 to 7;

FIG. 34 provides a partially exploded view of the vacuum unit of FIG. 33 to reveal the interface manifold of FIG. 24;

FIG. 35 provides an exploded view of the vacuum unit of FIG. 33;

FIG. 36 shows the vacuum unit of FIG. 33 with a top cover of an enclosure or housing of the unit removed to reveal components of the unit assembled inside the enclosure, with wiring and tubing connections omitted for clarity;

FIG. 37 provides a front view, side view and rear view a portable vacuum unit for the system of FIGS. 11 and 12;

FIG. 38 provides a rear view and a partially exploded view of the unit of FIG. 37 with a rear cover of an enclosure or housing of the unit removed to reveal the interface manifold of FIG. 19;

FIG. 39 is a schematic representation of the system of FIGS. 8 to 12 including an external wound treatment device;

FIG. 40 is a cross sectional view of the wound treatment device illustrated in FIG. 39;

FIG. 41 shows a supply and removal conduit arrangement to be provided together with a porting component in an external wound treatment device as shown in FIG. 40;

FIG. 42 shows an alternative supply and removal conduit arrangement to be provided together with a porting component in an external wound treatment device as shown in FIG. 40;

FIG. 43 illustrates various flow characteristics with air entrained in a flow of liquid;

FIG. 44 shows a system architecture for various embodiments of an NPT system described herein;

FIG. 45 shows a hardware architecture for various embodiments of an NPT system described herein;

FIG. 46 provides a high level control flow diagram for various embodiments of an NPT system described herein;

FIG. 47 provides a control flow diagram for an airflow state of the control flow diagrams of FIGS. 46 and 51;

FIG. 48 provides a control flow diagram for a pressurise state of the control flow diagrams of FIGS. 46 and 51;

FIG. 49 provides a control flow diagram for a hold pressure state of the control flow diagram of FIG. 46;

FIG. 50 provides a control flow diagram for a timeout state of the control flow diagrams of FIGS. 46 and 51;

FIG. 51 provides a high level control flow diagram for various embodiments of a NPWT system described herein;

FIG. 52 provides a control flow diagram for a hold pressure state of the control flow diagram of FIG. 51;

FIG. 53 provides a control flow diagram for a fluid flow state of the control flow diagram of FIG. 51;

FIG. 54 provides a control flow diagram for a flushing cycle of the fluid flow state of FIG. 53;

FIG. 55 provides a chart showing system performance of a treatment system test set-up;

FIG. 56 is a schematic representation of another treatment system including both an internal wound treatment device and an external wound treatment device;

FIG. 57 is a schematic representation of the system of FIG. 56;

FIG. 58 provides a high-level control flow diagram for various embodiments of an NPT system of FIGS. 56 and 57;

FIG. 59 provides a control flow diagram for a pressurise state of the control flow diagram of FIG. 46 or 58;

FIG. 60 provides a control flow diagram for a hold state of the control flow diagram of FIG. 46 or 58;

FIG. 61 provides a control flow diagram for an airflow state of the control flow diagram of FIG. 46 or 58;

FIG. 62 provides a control flow diagram for a timeout state of the control flow diagram of FIG. 46 or 58; and

FIG. 63 provides a control flow diagram for a dressing pressurise state of the control flow diagram of FIG. 46 or 58.

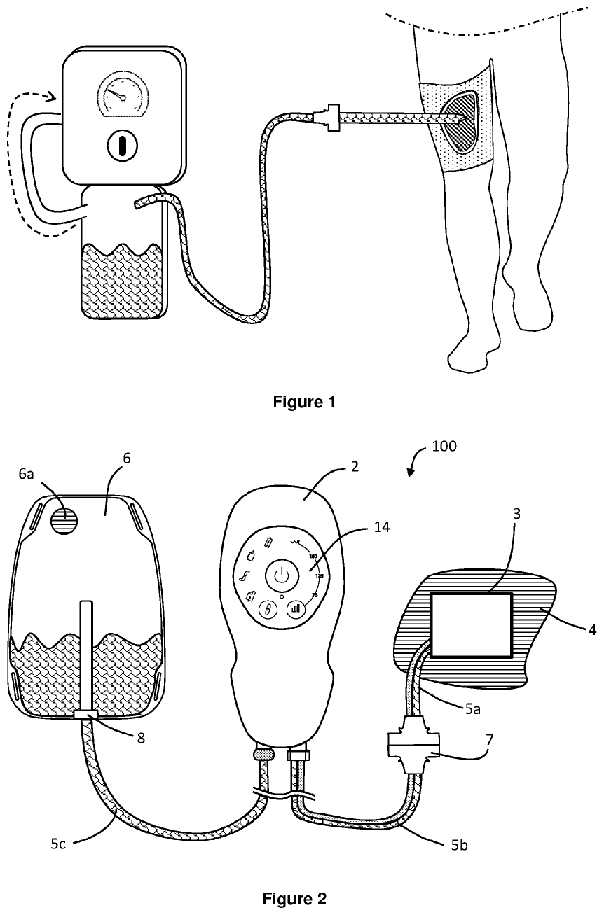

### DETAILED DESCRIPTION OF A PREFERRED EMBODIMENT

In the figures, like reference numbers are used for different embodiments to indicate like features.

FIGS. 2 to 12, FIG. 39, and FIGS. 56 and 57 show exemplary embodiments of negative pressure treatment systems (herein treatment systems) for the removal of fluid from a wound or for supplying treatment fluid to a wound and removing fluid from a wound.

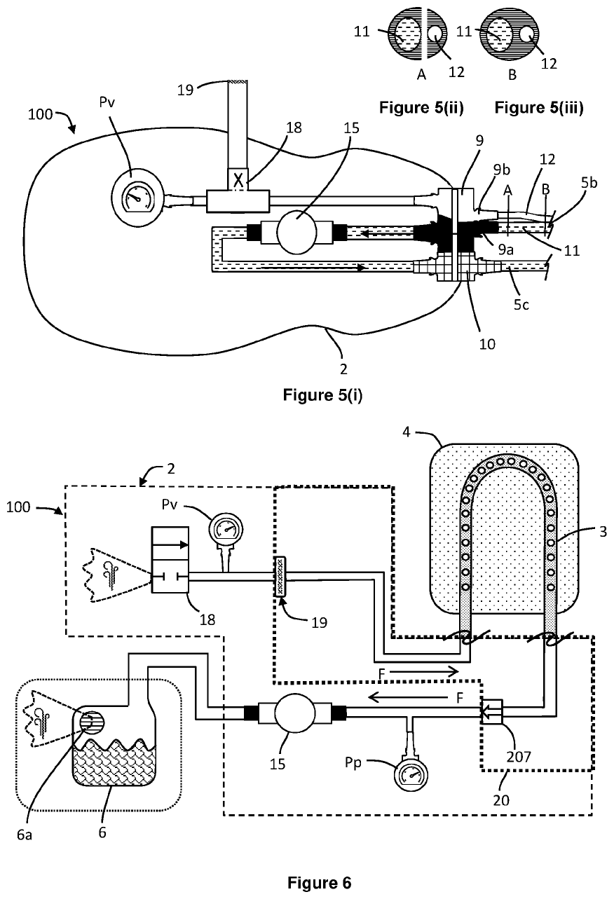

Referring to FIG. 2, at a general level the treatment system 100 comprises a wound treatment device 3 to be located at a wound treatment site (wound) 4, a vacuum pressure unit 2 comprising a vacuum pump assembly (for example pump assembly 15 in FIG. 5) for applying negative pressure to the wound 4 via the treatment device 3, and a fluid collection reservoir 6 for collecting fluid returned from the wound 4.

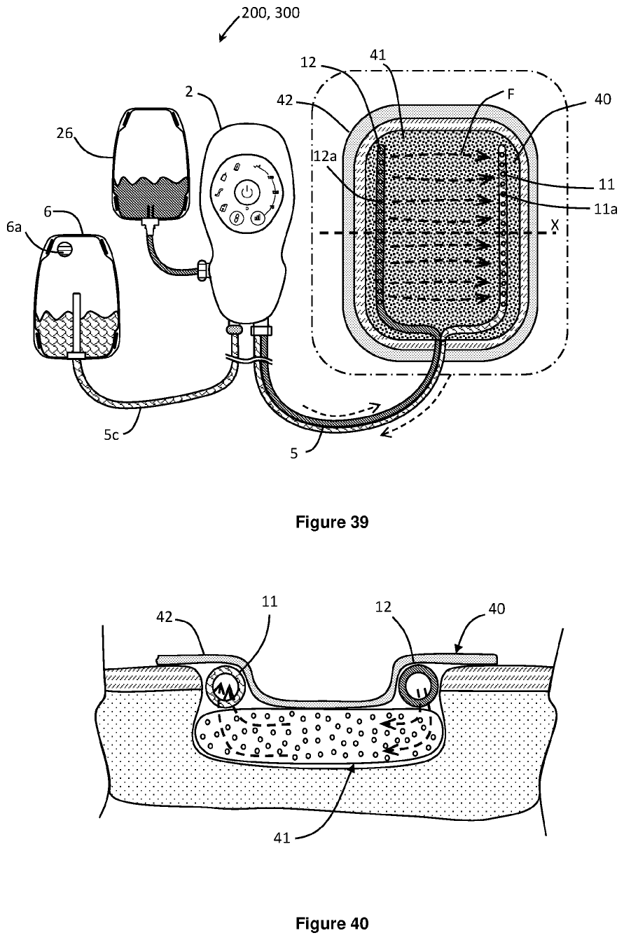

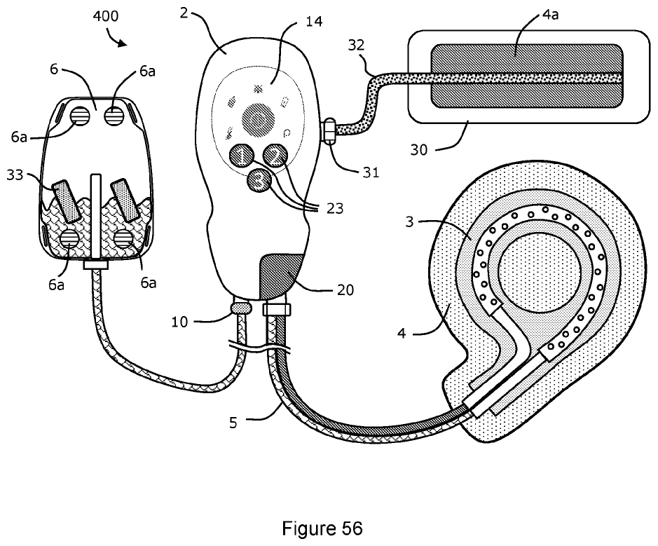

The vacuum pressure unit (or vacuum unit) 2 is configured to position the pump assembly 15 upstream of the fluid collection reservoir 6 and downstream of the wound treatment device 3. The wound treatment device 3 may comprise a topically applied wound dressing (FIG. 39), an implanted treatment device or a combination of both in a coupled configuration (FIG. 56). The fluid collection reservoir 6 is configured to include one or more air permeable filters or vents 6a to maintain the fluid collection reservoir 6 and connected conduit 5c at an ambient pressure level.

The vacuum unit 2 fluidly couples to the wound treatment device 3 via at least one conduit. The conduit from the vacuum unit 2 to the wound treatment device 3 may comprise a two-part conduit, with a first conduit 5b extending from the vacuum unit 2, and a second conduit 5a extending from the wound treatment device 3. The second conduit may be part of the wound treatment device 3 or may be connected to the treatment device 3 by a connector (not shown). A connector 7 is provided to fluidly couple the first and second conduits 5a, 5b. Alternatively, a continuous conduit may extend between the vacuum unit 2 and the treatment device 3.

Topically applied dressings for use in negative pressure wound therapy applications include a substantially air-impermeable and liquid impermeable occlusive layer that is adhered over the wound or incision to seal the wound site for the application of negative pressure. Typically, the conduit 5a extends from the dressing, but alternatively the dressing may have a connector to receive a conduit from the vacuum unit 2, or the occlusive layer may simply adhere and seal over the conduit.

The connector 7 may comprise a one-way valve oriented to allow fluid flow in a direction from the wound 4 towards the vacuum unit 2 and prevent a backflow of fluid from the pump to the wound. In alternative embodiments, a one-way valve may instead be provided within the vacuum unit 2, elsewhere on the conduit 5a, 5b, or as part of the treatment device 3. In a further alternative, the treatment system 100 may be without a one-way valve between the treatment device 3 and the vacuum unit.

In some embodiments, the conduit(s) between the vacuum unit 2 and the treatment device 3 may comprise a dual lumen conduit with a primary lumen for the passage of fluid flowing from the wound to the pump assembly 15, and a secondary lumen. The secondary lumen may allow for measurement of pressure at the wound site. The secondary lumen provides for the delivery of air and/or treatment fluids to the wound 4. However, in alternative embodiments multiple conduit(s) may be provided between the vacuum unit 2 and the treatment device 3 each with a single lumen.

A further conduit 5c is provided between the vacuum unit 2 and the reservoir 6 to fluidly couple the pump assembly 15 to the reservoir 6. A connector 8 may be provided to fluidly couple the conduit 5c to the reservoir 6.

In preferred embodiments, the vacuum unit 2 is a portable hand-held unit. The vacuum unit 2 may be a single use unit that is intended to used for a single patient. In an alternative embodiment the vacuum unit 2 could be configured for multi-patient use. The vacuum unit 2 comprises a (plastic) shell or enclosure to house the pump assembly 15 and other components. The vacuum unit 2 comprises a user interface 14 for operating the vacuum unit 2. The user interface may include controls to turn the pump assembly 15 of the system 100 on and off, and may allow an operator to control parameters of a pressure treatment being applied to the wound 4 such as the level of vacuum pressure being applied or the length, size and frequency of pressure oscillations between upper and lower set points.

In alternative embodiments the user interface 14 may also include controls to remotely connect a monitoring device to the vacuum unit to enable the transmission of data to an operator or user of the system to aid in the monitoring of treatment.

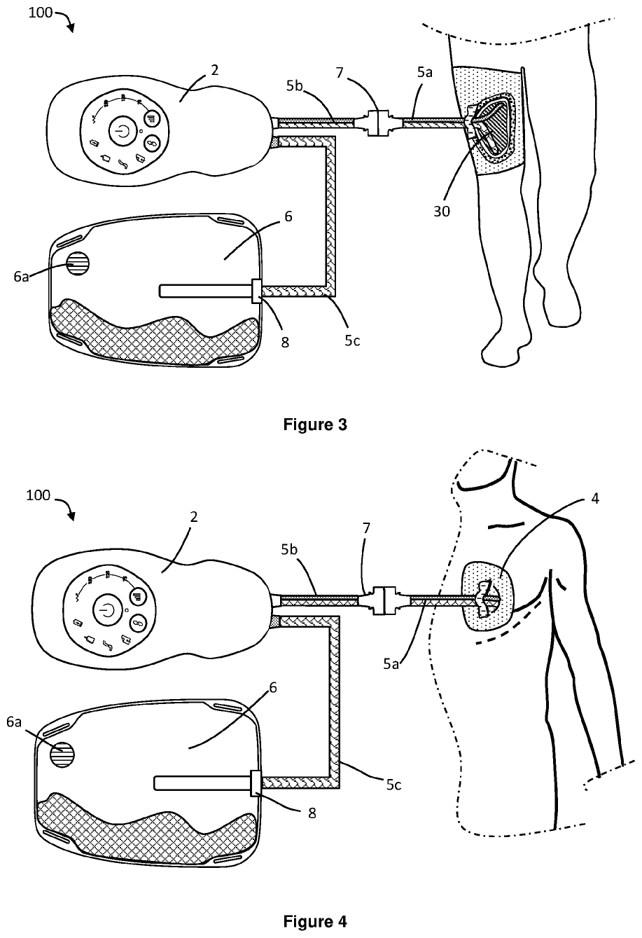

FIG. 3 illustrates the system 100 of FIG. 2 used with a dressing or external wound treatment device 30 for an open leg wound. However, the system 100 is also suitable for use for internal wound sites as illustrated in FIG. 4. FIG. 4 illustrates an internal wound site located at a chest area of a patient; however, the system may be used to treat internal wounds located at other sites for example to treat an abdominal wound.

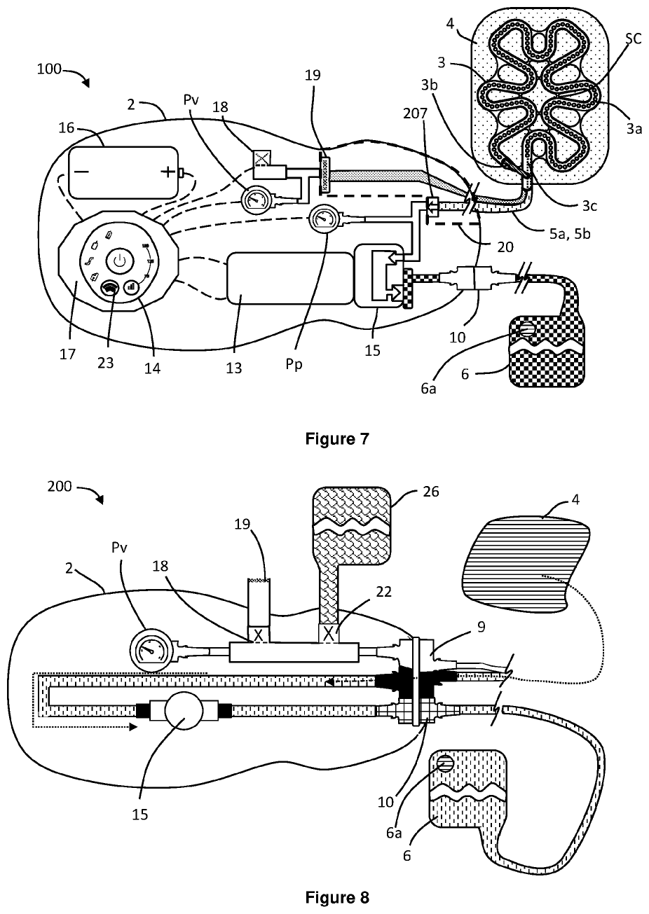

Referring now to FIGS. 5(i) to 5(iii) together with FIG. 2, the vacuum unit 2 comprises a housing or enclosure that houses a vacuum pump assembly 15 described in more detail below, batteries (16 in FIG. 7) or other power supply, a vacuum unit connector 9 in fluid communication with the conduit(s) 5b, 5a to deliver and receive fluid from the wound treatment site 4, and a vacuum unit outlet connector 10 in fluid communication with the conduit 5c to the reservoir 6, for the fluid flow from the pump assembly 15 to the reservoir 6. The connectors 9, 10 are configured to couple with ends of respective conduits 5b, 5c and may be of any suitable form, for example, they may comprise luer-type connectors.

In one embodiment the vacuum unit connector 9 may comprise two one-way valves such that a one-way valve within the secondary connector 9b is oriented to allow the flow of fluids from an upstream source, such as ambient air that has been passed through a sterile filter (filter 19 in FIG. 6) or from a treatment fluid reservoir (reservoir 26 in FIG. 8), to the wound 4. The corresponding one-way valve within the primary connector 9a is oriented to allow the flow of fluid in a direction from the wound 4 towards the vacuum unit 2. In some embodiments the one-way valves within the primary 9a and secondary 9b connector may be configured to be closed when the vacuum unit connector 9 is disconnected from the vacuum unit 2. These valves are then subsequently opened to allow the passage of fluids when the vacuum unit connected 9 re-connected to the vacuum unit 2. Examples of known prior art connectors that possess such features include needle-free or needless connectors for use within IV applications, such as the BDR MaxPlus™ needle-free connectors, which only allow a passage of fluid once engaged with an appropriate leur-lock connector.

The conduit 5b for fluid flow into and out of the vacuum unit connector 9 is a dual lumen conduit with a primary lumen 11 and a secondary lumen 12. The connector 9 includes a primary connector 9a providing a fluid inlet to connect to the primary lumen 11, and a secondary connector 9b providing a fluid outlet to connect to the secondary lumen 12 while keeping the flow from these lumens separated. The larger primary lumen 11 allows the passage of fluid flowing from the wound, through the primary connector, to the vacuum pump assembly 15. The secondary or supply connector 9b may be separate from the primary or removal connector 9a.

The primary and secondary lumens 11, 12 are preferably provided as adjacent passages in a single body/conduit along most of their length, as illustrated in the cross sectional view of FIG. 5(iii) taken on section line B in FIG. 5(i). However, adjacent the vacuum unit 2 and/or adjacent the wound treatment device 3, the dual lumen conduit 5a, 5b may be split or separated into two separate limbs or conduits, a supply conduit comprising the secondary lumen 12 and a removal or exudate conduit comprising the primary lumen 11 as shown in the cross sectional view of FIG. 5(ii) taken on section line A in FIG. 5(i), for ease of coupling to the vacuum unit 2 and/or to allow the supply conduit to enter the wound or wound treatment device 3 at a different location to the removal conduit. The primary or removal conduit and lumen may be referred to interchangeably and referenced by reference numeral 11 and the secondary or supply conduit and lumen may be referred to interchangeably and referenced by reference numeral 12.

The supply conduit 12 is in fluid communication with a pressure sensor Pv to allow for measurement of pressure on an upstream side of the wound treatment device 3.

The vacuum unit 2 comprises an air inlet valve 18 in fluid communication with the supply conduit 12. The air inlet valve 18 is controlled in a manner to introduce air into the treatment system 100 to assist with lifting fluid from the wound site 4, as described in more detail below.

As shown in FIG. 5(i), the air inlet valve 18 may have an inlet to draw ambient air to the system from outside the vacuum unit 2 enclosure. Alternatively, the inlet for the air inlet valve may take air from inside the vacuum unit housing/enclosure.

A sterile filter 19 is provided to prevent the ingress of bioburden and non-sterile air into the system 100 and wound site 4. In FIG. 5(i) the filter 19 is provided on an inlet of the air inlet valve 18, however a filter may be placed at another location between the air inlet valve 18 and the vacuum unit fluid supply connector 9b, or between the air inlet valve 18 and the wound site 4.

FIG. 6 illustrates the treatment system 100 schematically in more detail. The boundary or outer enclosure of the vacuum unit 2 is illustrated by the dashed line in FIG. 6. On an upstream side of the treatment device 3 the vacuum unit 2 comprises the air inlet valve 18, optionally the pressure sensor Pv and the sterile filter 19, and on a downstream side of the treatment device 3 the vacuum unit 2 comprises the pump assembly 15 and optionally a pressure sensor Pp between the pump assembly 15 and treatment device 3. The vacuum unit 2 may also comprise a connection manifold 20 providing a connection interface between the conduit 5a, 5b to the treatment device 3 and the vacuum unit 2. The connection manifold 20 is illustrated by the dotted line in FIG. 6 and the dashed line in FIG. 7 and replaces connector 9 shown in FIG. 5. The manifold is described in more detail below.

FIG. 7 provides a further schematic representation of the treatment system 100 equivalent to FIG. 6 but additionally illustrates the vacuum unit 2 comprising a power supply (in the form of a battery or battery pack 16), the user interface 14, a controller 17, and a drive motor 13 for driving the pump assembly 15. The motor, pressure sensors Pv, Pp and an actuator (not shown) for driving the air inlet valve 18 are in electrical communication with the controller 17. In this embodiment the pump controller 17 includes either a short wave or long wave form of wireless transmission via suitable electronics components which are known to those skilled in the art.

FIGS. 8 to 12 illustrate further embodiments of a treatment system for supplying fluid to and removing fluid from a wound. The embodiments of FIGS. 8 to 12 include the same or similar features of the system 100 described above with reference to FIGS. 2 to 7, however are additionally configured to provide a treatment fluid to the wound treatment device 3.

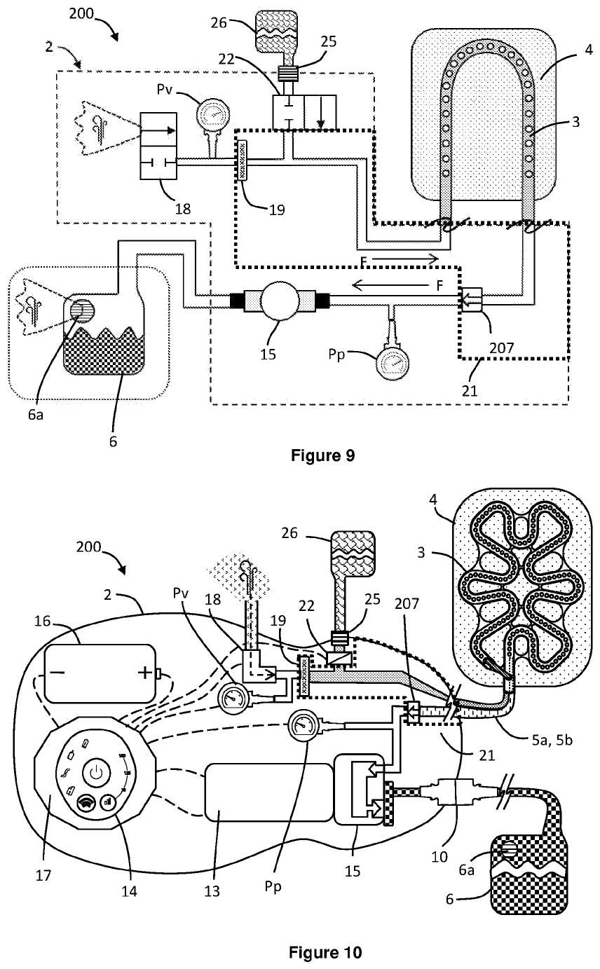

With reference to FIGS. 9 to 12, the vacuum unit 2 may comprise one or more ports 25 to receive therapeutic fluids for delivery to the wound site. The port 25 is preferably configured to be nominally closed to the passage of liquids when disconnected from the treatment fluid reservoir 26 which subsequently opens when engaged with a leur connector. The B. Braun Medical® CARESITE™ needless connector provides an example of such a port.

A therapeutic agent in the form of a treatment fluid may be selectively delivered to the wound treatment device 3 via the supply conduit 12. A fluid source or treatment fluid reservoir 26 may be coupled to the fluid port 25 of the vacuum unit 2, for example via a conduit or connection to an intravenous (IV) fluid giving set such as a Baxter® EMC 9608 Admin Set, B. Braun Medical® Single Chamber IV Infusion Set or similar sterile IV infusion therapy set. The treatment fluid reservoir is preferably at atmospheric pressure whilst connected to the treatment system. This can be achieved by using a non-vented IV infusion therapy set in combination with a flexible fluid bag such as Baxter® Sodium Lactate (Hartmanns or compound sodium lactate) IV Bag or similar, or it may also be achieved by connecting a vented IV infusion therapy set to a rigid or semi-rigid container of treatment fluid, such as Prontosan® Wound Irrigation Solution by B. Braun Medical®.

Example therapeutic fluids include, but are not limited to, compound sodium lactate, physiological saline (0.9% NaCL—Sodium Chloride) and 0.45% normal saline (0.45NaCL). Antimicrobial agents and solutions could also be applied for the treatment of infections and may contain agents such as polyhexanide (PHMB), silver nitrate, hypochlorous acid (HOCl), sodium hypochloride, betaine, sodium hypochlorite, super-oxidized water with neutral pH or any other antimicrobial wound irrigation solutions.

Other treatment fluids may also include cell-suspensions and cell-based fluids for promoting wound healing. The fluid may comprise flowable gels derived from ECM and mixed with water for injections, hyaluronic acid, growth factors to aid healing, to analgesic drugs such as fentanyl or morphine for pain relief and anti-inflammatory drugs such as ketorolac or diclofenac, for example, although other fluids are envisaged and will be apparent to a skilled person.

Instillation of autologous or allogenic cell-based therapies containing either platelet rich plasma, stem cells, stromal cells, keratinocytes, lymphocytes, bone marrow aspirate, serum and dendritic cells could aid in the repair and healing of wounds.

The instillation of chemotherapeutic drugs could also aid in the localised treatment of cancerous cells that may not be operable, or could be used as an overall treatment plan following excision of cancerous tissue.

With reference to the embodiment 200 of FIGS. 9 and 10, a treatment fluid inlet valve 22 is selectively operable to allow fluid to flow from the treatment fluid reservoir 26 and into the supply conduit 12 leading to the wound. The reservoir of fluid is at atmospheric pressure. When the treatment fluid inlet valve 22 is selectively opened, negative pressure from the pump assembly 15 applied to the wound 4 via the removal conduit 11 acts to draw fluid from the treatment fluid reservoir 26 towards the dressing or wound treatment device 3. Upon activation of the treatment fluid inlet valve 22 the controller (not shown in this figure) within the vacuum unit 2 detects a subsequent drop in the vacuum pressure level at the Pv and/or Pp pressure sensor(s) and activates the pump assembly 15 to maintain the vacuum pressure at a target level of vacuum pressure. A control algorithm is described in more detail below. In the illustrated embodiment, the air inlet valve 18 and sterile filter 19 is provided upstream of the therapeutic fluid valve 22.

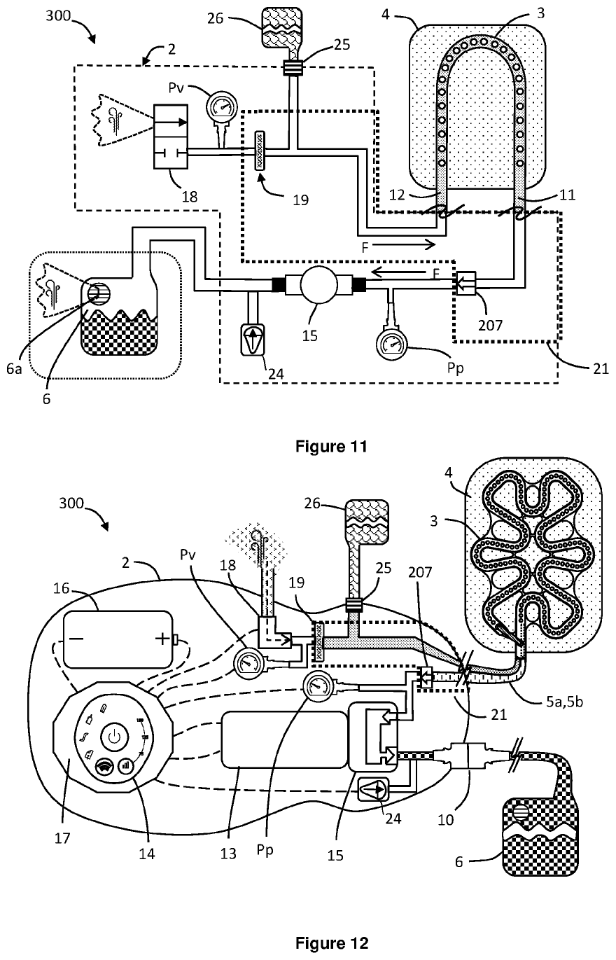

In the embodiment 300 of FIGS. 11 and 12, the system is without a treatment fluid inlet valve 22. The system 300 may include an orifice or other flow restriction to control an amount of treatment fluid introduced to the system during negative pressure treatment. In one embodiment the administration of treatment fluids is controlled via the use of an intravenous (IV) fluid giving set such as a Baxter® EMC 9608 Admin Set, B. Braun Medical® Single Chamber IV Infusion Set or similar sterile IV infusion therapy set which is connected to the unit 2 via the fluid port 25. The fluid flow rate of treatment fluid being introduced to the supply conduit 12 is controlled via a roller clamp in the set, which is adjusted to vary the flow restriction within the section of tube that interfaces with the roller clamp component. In this embodiment the rate of fluid instillation can be visually checked via the drip chamber of the IV infusion set when the chamber is orientated upright, with any further flow adjustments made via the roller clamp adjustment. This embodiment provides a manual means to introduce a treatment fluid to the wound 4 via the wound treatment device 3.

In an alternative embodiment the vacuum unit 2 may be connected to an infusion pump via the fluid port 25 to allow fluids to be supplied to the wound treatment device 3 in a selectable and controllable manner. Such infusion pump systems could include the B. Braun Medical® Vista® basic large volume infusion pump or the BDR Alaris® Syringe Module for example, which can controllably deliver from 0.1 ml/hour to 1200 ml/hour of treatment fluid on either an intermittent or constant fluid delivery basis. These systems typically offer the means to select the amount, flow rate and frequency of which treatment fluid is dispensed. When treatment fluid is introduced into the vacuum unit 2 the system detects the subsequent drop in the set vacuum pressure level at the Pv and/or Pp pressure sensor(s) and activates the pump assembly 15 to maintain the systems target level of vacuum pressure. A control algorithm is described in more detail below.

In the embodiments of FIGS. 8 to 12, the vacuum unit 2 comprises a connection manifold 21 providing a connection interface between the conduit 5a, 5b to the treatment device 3 and the vacuum unit 2 and between the vacuum unit 2 and the treatment fluid reservoir 26 via the fluid port 25. The connection manifold 21 is illustrated by the dotted line in FIGS. 9 to 12 and replaces connector 9 shown in FIG. 8. The manifold is described in more detail below.

In the embodiment system 300 of FIGS. 11 and 12 the vacuum unit 2 additionally includes a colour sensor 24 that is electronically connected to the vacuum unit controller 17. In this embodiment 300, the colour sensor 24 is positioned along the fluid flow path positioned between the outlet of the pump 15 and the outlet connector 10. However, the colour sensor could alternatively be positioned along the fluid pathway in any suitable position upstream of the inlet of the pump 15.

The colour sensor 24 may be beneficial to detect a colour change of wound exudate fluid flowing through the system from the treatment device 3 at the wound site 4. For example, the natural change in colour from a first blood rich wound exudate immediately following surgery, to a pink colour of serosanguinous drainage (blood and serum), and/or to a clear serous (serum only) drainage. This operation of the colour sensor 24 may be enhanced by the supply of filtered air from upstream of the treatment device 3. The filtered air displaces the fluid to produce a readable sample of fluid within that short time frame, similar to that of a direct aspiration of fluid from the treatment site 4 via a needle.

The inclusion of a colour sensor within various embodiment systems that supply treatment fluid to, and remove treatment fluid from, the wound may offer further benefits. For example, the colour sensor 24 could be configured to detect the passage of treatment fluid being supplied from the treatment fluid reservoir 26 and passing through the upstream fluid pathway, removal conduit 11, wound treatment device 3 and supply conduit 12, to the vacuum unit 2 denoting the complete saturation of treatment fluid through the connected system. In other embodiments the treatment fluid could be combined with a colour based indicator for the detection of changes at the wound in response to the presence of infection, biofilm or other wound based pathologies.

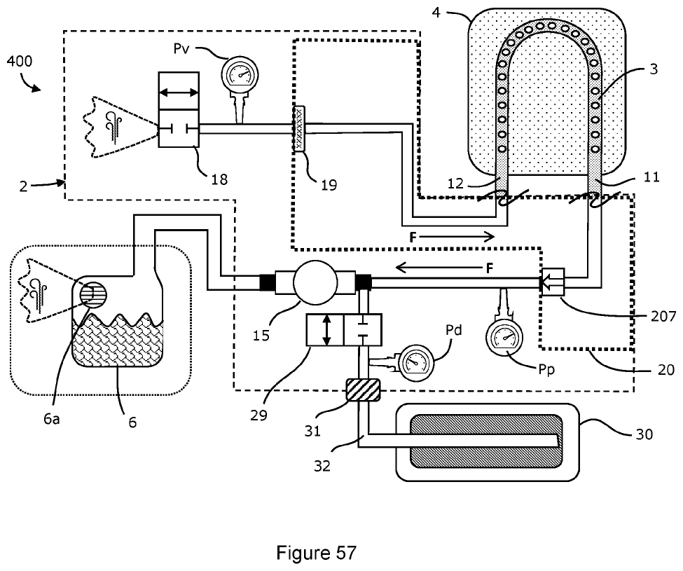

The embodiment treatment system 400 of FIGS. 56 and 57 is similar to the embodiment 100 of FIG. 6 but includes an additional control valve 29 for coupling to a secondary wound treatment device. The control valve 29 is coupled to and in fluid communication with to the inlet of the pump 15. In the configuration shown in FIG. 57 an external wound treatment device 30 is connected to the vacuum unit 2 via a further fluid conduit 32 to the primary lumen 11 coupling the primary wound treatment device 3 to the outlet of the pump 15 via the manifold 20. In the embodiment 400, the vacuum unit 2 includes a dressing port 31 to connect the external wound treatment device 30 to the inlet of the pump 15 via a conduit 32. Delivery of vacuum pressure to the external wound treatment device 30 is controlled via an actuator of the dressing pressure control valve 29.

In the embodiment 400 shown in FIG. 57, the controller 17 of the system is connected to the dressing pressure sensor Pd that is positioned upstream of the pump 15 and dressing pressure control valve 29 and downstream of the dressing port 31, with the controller configured to supply vacuum pressure to the wound treatment device 30 from the pump 15. The controller of this embodiment system is described in more detail below.

Various components of the treatment systems 100, 200, 300, 400 are now described.

### Reservoir

As described, the treatment system 100, 200, 300, 400 comprises a reservoir 6 for collecting liquids removed from the wound site 4, for example, wound exudate. In a preferred embodiment, the reservoir 6 is positioned at the furthermost position away from the wound and therefore is downstream of the pump assembly 15, for collecting fluids removed from the wound after they have passed through the pump assembly 15.

In the embodiments shown, the reservoir 6 comprises a flexible bag. Alternatively, a rigid reservoir could be provided.

The reservoir 6 comprises one or more air permeable filters or vents 6a provided in a wall of the reservoir, for example a hydrophobic venting membrane provided over an aperture in the impermeable membrane. The air-permeable filter(s) or vents(s) allow the venting of gases and thereby prevent pressure build-up in the reservoir preventing effective pumping. An example reservoir has eight vents 6a each having an 8 mm diameter and a pore size of 3 micron to sustain a high level of airflow passing through the system.

Blood clots, fibrin and other solidified fluids or tissue debris may block the venting membranes which causes the bag to inflate with air introduced to the fluid path. This inflation can cause the bag to pop and leak fluid or can prohibit the pump from generating vacuum pressure required by forcing the outlet valves from opening under excess positive pressure.

To avoid these issues a high salt compatible sodium polyacrylate polymer, or other equivalent blood compatible superabsorbent polymers may be added to the reservoir to solidify the blood and wound fluid in the bag. These polymers are available either as loose particles, particles suspended within a dissolvable PVA film pouch or polymer suspended within a textile/fabric like medium. In the embodiment shown in FIG. 56 the reservoir 6 is shown comprising two bags of absorbent polymer 33.

The use of this polymer in tandem with one or more vents on the bag avoids bag inflation and allows the fluid path of the treatment system to cope with much more air as it is introduced into the system.

### Pump Assembly

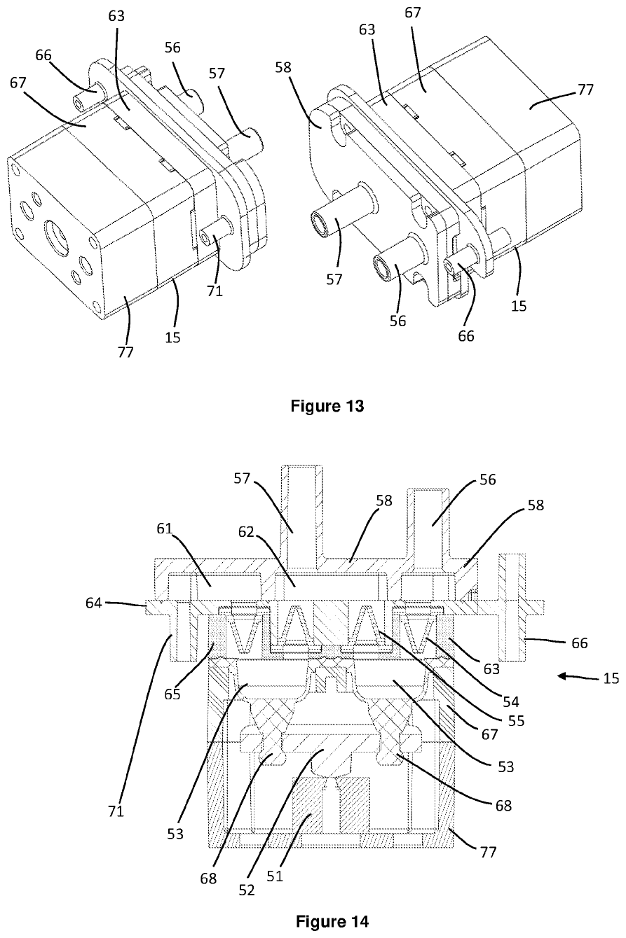

The vacuum pump assembly 15 will now be described with reference to FIGS. 13 to 16. The pump assembly 15 is driven by the motor 13 as illustrated in FIGS. 7, 10 and 12. The pump assembly 15 comprises a swash plate 52 a plurality of flexible chambers 53 (diaphragms), a plurality of pairs of flexible valves 54, 55, each pair of valves being in fluid communication with a respective flexible chamber 53, and a pump inlet 56 and outlet 57.

### Pump Cover/Inlet/Outlet

The pump inlet 56 and outlet 57 are provided on a pump cover 58. In the embodiment shown, the inlet 56 and outlet 57 are provided side-by-side on the pump cover 58, with the inlet 56 situated nearer an edge of the pump cover 58 and the outlet 57 positioned nearer the centre of the pump cover 58.

Each of the inlet and outlet 56, 57, comprises an aperture that extends through the pump cover for the passage of fluid into and out of the pump, respectively. Referring to the exploded view of FIG. 16, the underside of the pump cover 58 comprises two channels—an (outer) inlet channel 61, surrounding an (inner) outlet channel 62. The aperture from the inlet 56 opens into the inlet channel 61 such that fluid flowing into the pump through the inlet 56 flows into the inlet channel 61. The aperture from the outlet 57 opens into the outlet channel 62 such that fluid flowing out of the pump flows into the outlet channel 62 and out through the outlet 57.

The inlet channel 61 and the outlet channel 62 are distinct and fluidly separate such that fluid cannot flow directly from one channel to the other.

### Valves

Referring to FIGS. 14 to 16, the pump comprises two pairs of valves, each valve pair corresponding with and being aligned with a respective chamber 53. Each valve pair consists of an inlet valve 54 and an outlet valve 55. The valve support component positions the inlet valves 54 to be in aligned with the inlet channel 61 such that fluid from the inlet channel is in fluid communication with the inlet valves 54. The outlet valves 55 are each positioned to be aligned with and in fluid communication with the outlet channel 62 such that fluid from the outlet valves 55 will flow into the outlet channel 62.

The valves 54, 55 are one-way valves to allow fluid through the valve in one direction and to prevent fluid flowing through the valve in the opposite direction. In each valve pair, the inlet valve and outlet valve 54, 55 are oppositely oriented so that fluid can only flow into the corresponding chamber 53 through the respective inlet valve 54 and out of the chamber 53 through the respective outlet valve 55.

The valves 54, 55 each comprise a resilient “duck-bill” type valve. These duck-bill valves each have two oppositely inclined walls, with a single slit-type opening at the apex of the two walls. Under pressure from fluid between the two walls, the slit is forced open, by the two walls moving apart, to allow fluid flow through the valve.

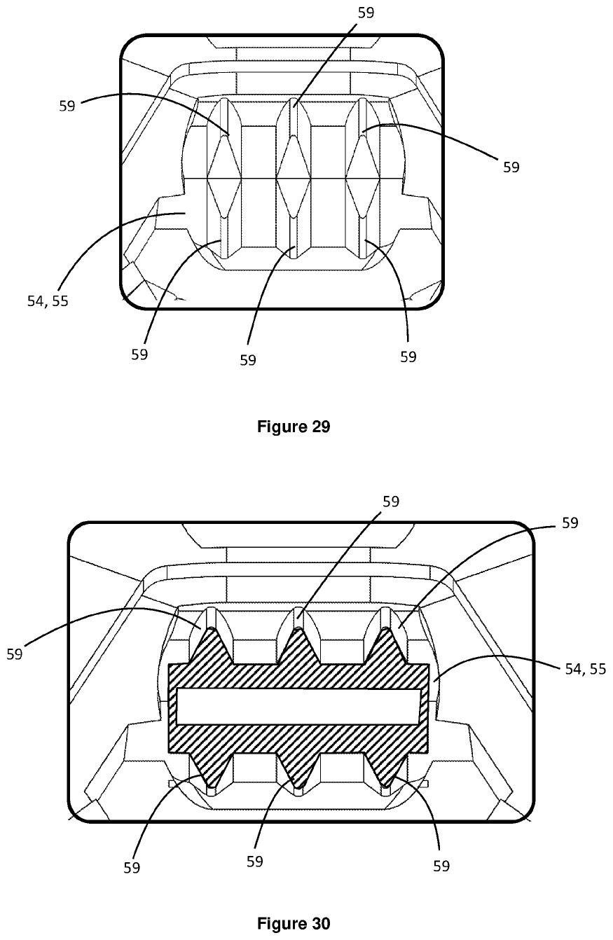

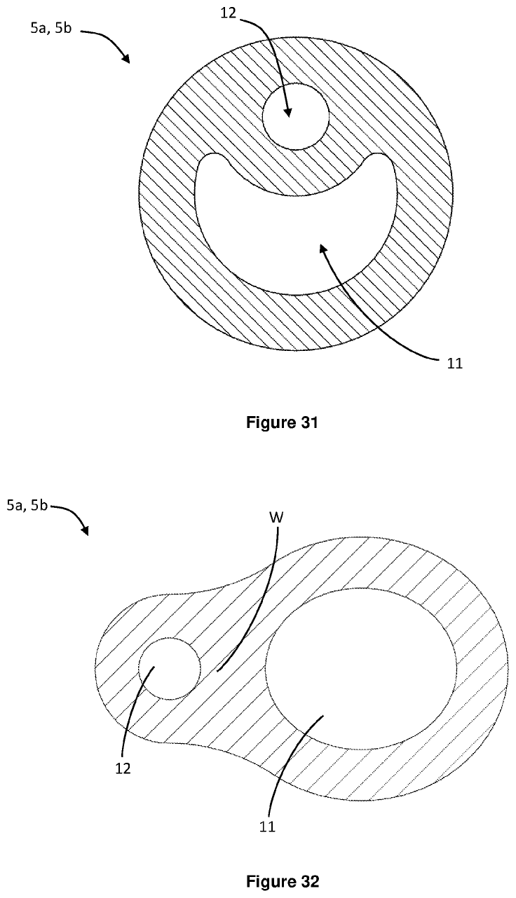

With reference to FIGS. 29 and 30, in some embodiments, each flexible valve 54, 55 may comprise a plurality of stiffening ribs 59 on the downstream surface of the inclined walls. These ribs 59 help maintain the valve shape under back pressure, to reduce the chance of the valve collapsing, folding, or inverting under back pressure, as a thin-wall flexible valve may otherwise be susceptible to. The ribs 59 provide stiffness to the walls without inhibiting the flexing and opening of the valve under flow. In the embodiment shown, the ribs are substantially triangular in cross section, but in alternative embodiments they may have other shapes. The stiffening ribs may also stiffen the valve to reduce the occurrence of leaks in a reverse direction through the valve.

With reference to FIGS. 13 to 16, the valves 54, 55 are supported in a valve housing 63. The valve housing comprises two parts 64 and 65. The two parts are fixed together with the valves 54, 55 retained in between the two parts 64, 65. Each part of the valve housing may be described as a valve support component. A flange portion of the valves 54, 55 is compressed between the two valve housing parts 64, 65 to provide a seal and prevent leaks. The valve housing 63 supports the valves 54, 55 so that the inlet valves 54 are in fluid communication with the inlet channel 61 and the outlet valves 55 are in fluid communication with the outlet channel 62 and with the valves 54, 55 arranged pairs to correspond with a single chamber 53 as described above.

The valve housing 63 is secured to the pump cover 58 to be fluidly sealed with the pump cover and separate the inner and outer channels 61, 62 and therefore pump inlet 56 and outlet 57. For example, one part 64 of the valve housing 63 is ultrasonically welded to the pump cover 58. The entire pump assembly 15 is then fastened to together using screws to clamp the valves 54, 55 within the valve housing 63.

The pump assembly 15 comprises a fluid flow path through the pump from the pump inlet 56 to the pump outlet 57 via the inlet valves 54, chambers 53, and outlet valves 55. In the illustrated embodiments the inlet and outlet valves 54, 55 each present a single orifice in a flow path when open.

As described above, in a preferred embodiment the exudate reservoir 6 is downstream of the pump assembly 15. This means fluid from the wound passes through the pump assembly 15. A valve 54, 55 presenting a single orifice reduces the risk of blockages in the pump assembly 15 caused by debris such as tissue debris, fibrin, blood clots, loose connective tissue and adipose (fat) tissue returned from the wound 4 passing through and blocking the vacuum pump assembly. Other valve types such as umbrella valves comprise a plurality of smaller apertures and are therefore more prone to developing blockages at the valve.

The single orifice of each valve 54, 55 has an area when the valve is open similar to or greater than a minimum area of the fluid flow path between the pump inlet 56 to the pump outlet 57. Preferably the open area of the single orifice of each valve 54, 55 is equal to or greater than an area of the pump inlet 56. Therefore, if a blockage was to occur at the pump assembly, this would occur at the pump inlet 56, not at a point inside the pump assembly. Preferably the open area of a single orifice is greater than a cross sectional area of the lumen of the supply conduit 11.

The illustrated embodiment includes duck bill valves 54, 55. However, other valves presenting a single large orifice to the pump flow path may be possible, such as a flapper valve, scupper valve, check valve, cross-slit valve and a dome valve. However, a valve consisting of a single unitary flexible member is preferred. The valves are preferably moulded from liquid silicone rubber (LSR) to reduce the likelihood of protein from the wound, such as fibrin, binding to the valve.

In the illustrated embodiment the valve housing 63 comprises a through port 66 with opposed spigots for attaching hoses to conveniently secure a fluid lumen separate from a fluid flow path through the pump assembly 15. The through port 66 and spigots may be provided elsewhere on the pump assembly, for example as part of the pump cover 58, or the pump assembly may be without the through bore and spigots. The illustrated embodiment includes a port 71 for connecting a pressure sensor (Pp), for example via a tube, for measuring the pressure in the inlet channel of the pump indicative of the system pressure downstream of the treatment device.

In other embodiments, the port 71 may be configured for connecting a control valve, for example the dressing control valve 29 in the embodiment 400 of FIGS. 56 and 57, to the pump inlet in order to supply vacuum pressure to a secondary wound treatment device 30. Alternatively, or additionally, the pump cover 58 could include one or more further ports 71 to facilitate connection with a pressure sensor and a valve.

### Cylinders/Pistons Swash Plate

Each pair of valves 54, 55 is in fluid communication with a respective flexible chamber 53. The embodiment shown comprises two chambers 53, corresponding to the two pairs of valves 54, 55. However, alternative embodiments may have a single chamber or more than two chambers. Preferably the pump assembly 15 comprises two or more chambers 53 and associated pairs of valves 54, 55 such that there is always one chamber compressing and one chamber expanding.

In the illustrated embodiment, the chambers 53 are provided integrally as a single component. The component comprises a flexible, resilient and air impermeable material such as silicone. A chamber housing 67 supports and houses the chamber component and attaches to the valve support housing 63 to hold the chambers 53 in alignment with the respective valves 54, 55.

The flexible chambers 53 are substantially cylindrical. Each chamber comprises an associated connector 68 projecting from an underside of the chamber 53 that is movable axially (along the axis of the cylinder), to compress and extend the chamber 53.

In the illustrated embodiment the connectors 68 connect to a swash plate 52, which has attachment features for attaching to the chamber connectors 68.

The swash plate 52 is driven by the motor 13 (not shown in these figures) via a rotational coupler 51. The coupler 51 is fixed to a drive shaft of the motor 13 such that the coupler 51 rotates together with the drive shaft about a drive axis. The coupler 51 has a mounting aperture 70 offset from the axis of rotation of the coupler 51.

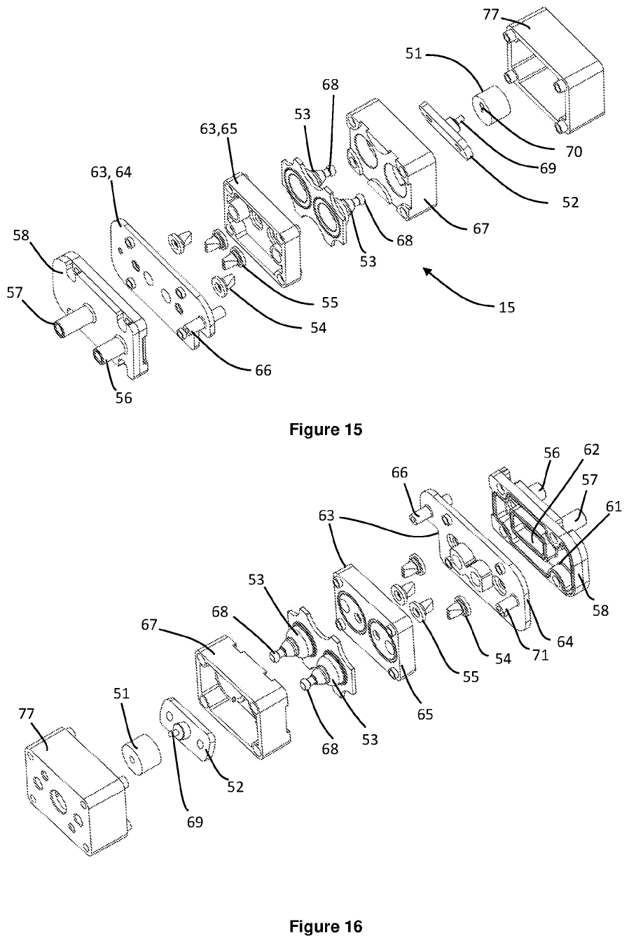

Referring to FIGS. 15 and 16, the swash plate 52 has a central spigot 69 that is received by the offset aperture 70 on the coupler 51. This causes the swashplate 52 to tilt as sideways movement of the swashplate 52 is constrained by the connections with the chambers 53 and the chamber housing 67.

The spigot 69 is pivotally mounted within the offset aperture 70 of the coupler 51 such that the coupler 51 can rotate relative to the swash plate 69. As the coupler 51 is driven to rotate by the drive shaft of the motor 13, the end of the spigot 69 mounted in the coupler 51 moves in a circle around the drive axis, causing the swash plate 52 to tilt cyclically and axially in a seesaw fashion.

As the swash plate 52 cyclically tilts, it compresses each chamber 53 in turn and subsequently expands each chamber 53 in turn. The compression of a chamber 53 causes fluid present in the chamber 53 to be expelled through the respective outlet valve 55, into the outlet channel 62, and through the pump outlet 57. Subsequent expansion of the chamber 53 creates a vacuum within the chamber 53, drawing fluid from the pump inlet 56 through the inlet channel 61 and the respective inlet valve 54 and into the chamber 53. This process cyclically repeats to pump fluid from the pump inlet 56 to the pump outlet 57.

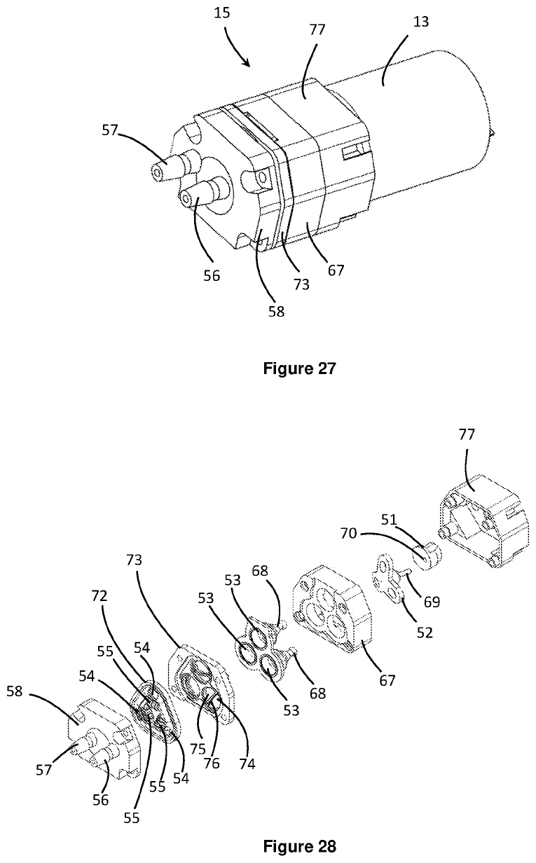

The motor 13, coupler 51 and swash plate 52 form a drive mechanism for driving expansion and compression of the chambers 53. The swash plate 52 and coupler 51 covert rotational motion of the motor 13 into axial motion to expand and compress the chambers 53. Other drive mechanisms are possible, for example a crank arm attached to the motor shaft and a connecting rod between the chamber and crank arm. However, where there are two or more chambers, a motor with coupler and swash plate is a preferred drive mechanism. In the illustrated embodiment of FIGS. 13 to 16, the swash plate 52 and coupler 51 are housed in a drive mechanism housing 77. The motor 13 may be mounted to the drive mechanism housing 77 (for example as shown in the embodiment of FIG. 27).

FIGS. 27 and 28 show an alternative pump arrangement comprising three chambers 53 and three associated pairs of inlet and outlet valves 54, 55. The plurality of pairs of flexible valves 54, 55 are provided integrally by a valve component 72. The valve component 72 comprises a flexible, resilient material such as silicone, and is mounted on a substantially rigid valve support 73. The valve support 73 comprises a recess that has a shape corresponding to the valve component 72 to locate and receive the valve component 72. The valve support 73 further comprises a plurality of pairs of apertures 74, 75, corresponding to each valve pair 54, 55. The two apertures 74, 75 in each pair is separated by a sealing bar 76. As shown in FIG. 28, the valve component 72 sits between the pump cover 58 and the valve support component 73 and is seated on the valve support 73 with the inlet valves 54 projecting into the corresponding aperture 74 in the valve support 73. The valve component 72 may comprise a locating flange shaped and positioned to abut the edge of the respective aperture to help to locate the valve 54, 55 and prevent leaks through the assembly during operation.

The sealing bars 76 are positioned between respective inlet and outlet valves 54, 55. The sealing bars 76 align with a portion of the pump cover that separates the inlet channel 61 and the outlet channel 62 of the pump cover 58. When the components are assembled, the sealing bars 76 bear against the valve component between the two valves 54, 55, compressing the valve component at that point to form a seal and prevent fluid bypassing the valves 54, 55 and flowing directly between the inlet and outlet channels 61, 62.

In the embodiment of FIGS. 27 and 28, the swashplate 52 has a Y-shape with three arms for connecting to the respective three compressible chambers 53. This Y-shape provides optimal clearance in this embodiment between the swash plate and the other components; however, the swashplate may have an alternative shape depending on the number of connectors it is driving, or it may have a substantially round shape.

The preferred described pump configurations described above with reference to FIGS. 13 to 16 and 27 and 28 achieve a high flow capacity at a low power usage, making the pump particularly useful for a NPT system, and in particular portable NPT systems.

For example, the pump described with reference to FIGS. 13 to 16 comprising two chambers has the pump characteristics as set out in Table 1. For comparison, the characteristics of a peristaltic pump is provided in Table 2 below. The pump described herein has a much higher flow capacity for a given power consumption. For example, at 6V the described pump has a flow rate of 220 ml/min at a power consumption of 0.33 W, whereas the peristaltic pump has a flow rate 38 ml/min at a power consumption of 0.9 W.

TABLE 1
pump described above with reference to FIGS. 13 to 16:

| Voltage (V) | L/min | Current (mA) | Power (W) |
|---|---|---|---|
| 3.3 | 0.11 | 45.6 | 0.15 |
| 5 | 0.19 | 53.3 | 0.27 |
| 6 | 0.22 | 54.5 | 0.33 |
| 7.5 | 0.27 | 56.6 | 0.42 |

TABLE 2
peristaltic pump:

| Voltage (V) | L/min | Current (mA) | Power (W) |
|---|---|---|---|
| 3 | 0.015 | 45.6 | 0.48 |
| 6 | 0.038 | 53.3 | 0.9 |
| 12 | 0.033 | 54.5 | 3 |

The pump assembly 15 is particularly beneficial in a preferred NPT system 100, 200, 300, 400 in which the air inlet valve 18 is opened to introduce air while continuing to maintain negative pressure at the wound 4, as described in more detail below. Such a system operation requires a high capacity pump 15 in order to maintain a negative pressure while introducing significant volumes of air to the treatment system 100, 200, 300, 400 with the air inlet valve 18 open for a significant time portion of a valve open and close cycle time. Furthermore, the pump assembly 15 is particularly useful in a treatment system comprising a treatment device 3 configured to introduce filtered air to a large portion of the total volume of the treatment site 4. A preferred treatment device 3 described below with reference to FIGS. 39 and 40. A large capacity pump assembly 15 is required to move the increased amount of air and lift fluid from the wound 4 to the exudate reservoir 6 while continuing to maintain a negative pressure at the wound 4 at an effective negative treatment pressure level.

Prior art NPT systems configured with a vacuum pump assembly upstream of the fluid collection reservoir and downstream of the wound treatment device typically use peristaltic pumps, since they can cope with passing tissue debris and provide the benefit of a closed system and with fluid separated from direct contact with moving parts of the pump. However, a peristaltic pump provides insufficient capacity at a practical size and power to achieve the required negative treatment pressure and flow rates required in a preferred system configuration.

A peristaltic pump providing a suitable flow rate for the preferred system described herein would be unsuitable for portable systems due to its size and power requirements. The described pump assembly 15 allows for improved capacity (increased flow rate) at a lower power compared to prior art pumps, while enabling biological matter such as blood, adipose tissue, fibrin, lysed cells and large biological particles (2 mm in size) to pass through the pump assembly 15 without causing blockages.

The described pump assembly 15 may have utility in other applications requiring a pump with a high capacity output for a relatively low power input. For example, the described pump may be particularly suited for use in a portable dialysis device or in any other portable device where the movements of large volumes are required, particularly in applications that required the movement of large volumes at a pressure level above or below ambient levels.

### Sterile Filter

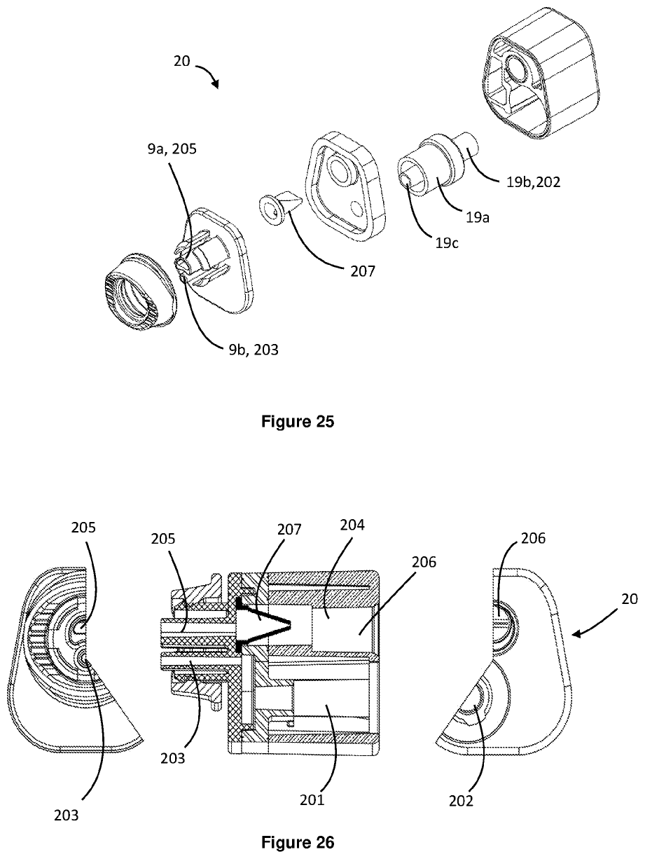

The sterile air inlet filter 19 may comprise a PTFE membrane, for example PTFE syringe type filters available from Steriltech™. In one example, the filter 19 comprises a filter membrane pore size of approximately 0.2 micron. The filter membrane may have an area of about 1 cm². The filter may comprise a filter assembly including a housing for enclosing a filter membrane or element and with an inlet and outlet (for example filter assembly 19 in FIG. 25 comprising housing 19a with inlet 19b and outlet 19c). A suitable filter is filter part number PT021350 provided by Steriltech™.

The filter 19 preferably additionally provides a predetermined pressure drop between the ambient pressure outside the treatment system 100, 200, 300, 400 and the pressure in the treatment system 100, 200, 300, 400 on the upstream side of the treatment device 3. The pressure drop may be provided by a filter membrane and/or an orifice in a flow path through a filter assembly. For example, the filter is chosen to give a pressure drop of about 20 to 130 mmHg. In an example embodiment, the filter provides a pressure drop of about 100 mmHg. Alternatively, a pressure drop between the ambient environment and an upstream side of the wound treatment device may be provided by another component, such as an orifice plate or other inlet restriction located in the system upstream of the wound treatment device. When the air inlet valve is open, the inlet restriction determines the pressure at the wound together with control of the pump assembly on the downstream side of the treatment device 3.

In particular embodiments where the secondary conduit 12 or supply pathway of the vacuum pressure unit 2 includes a common connection between the air inlet valve 18 and the treatment fluid reservoir 26, such as shown in the embodiments of FIGS. 8 to 12 the filter/filter element is preferably hydrophobic to prevent filter 19 blocking following contact with the treatment fluid from the treatment reservoir 26. Otherwise any type of suitable filter medium may be used.

The air filter 19 may be provided at an air inlet to the treatment system 100, 200, 300, 400, or within an air flow path of the treatment system. For example, the vacuum pressure unit enclosure may be hermetically sealed to prevent the unwanted ingress of fluid (such as water from showering or rain etc) into the vacuum unit 2 and provide a passage to the air inlet valve 18 via an exterior opening in the enclosure. In this instance a sterile filter membrane may be welded or otherwise attached to a port in the housing to ensure the air path to the wound is sterile and biocompatible. The downside of this is that all fluid contact parts of the system including the air inlet valve 18 must be sterilised.

In a preferred embodiment, the filter 19 is located between the air inlet valve 18 and the wound site as shown in FIGS. 6, 7, 9 to 12, and FIG. 57. This allows for a non-sterile air inlet valve assembly 18 to be incorporated into the system 100, 200, 300, 400 within the vacuum unit housing and draw ambient air into the valve 18 from the surrounding environment. Where an upstream pressure sensor Pv is provided, this is preferably upstream of the filter 19, so the sensor also does not require sterilisation.

### Air Inlet Valve and Fluid Inlet Valve

The air inlet valve 18 includes an actuator such as a solenoid in electrical communication with the controller to drive the valve between open and closed positions. An example of a suitable valve for use as the air inlet valve 18 is a mini solenoid valve provided by Koge™ part number KSV2WM-5A. This particular solenoid valve has a central ferromagnetic plunger component which remains nominally closed against an internal rubber seal via the force provided by an internal spring. This valve is opened through the application of an electrical current to generate a magnetic field which opens the plunger against the force of the spring. This valve has the advantage of automatically closing upon the loss of electrical current to preserve the level of vacuum pressure within the treatment device 3, which is advantageous when electrical power is unexpectantly lost, e.g. when the battery loses charge. The disadvantage of this valve is the total amount of energy required to keep the valve open for long durations.

Another example of a suitable air inlet valve 18 is the NLV-2-MFF micro latching solenoid diaphragm isolation valve provided by Takasago Fluidic Systems (Takasago Electric, Inc). This solenoid valve uses permanent magnets to maintain the valve in either the open or closed position. The supply of electrical current to the solenoid in a first direction will shift the valve from an open to a closed status, while the supply of electrical current in a second reverse direction will shift the valve from an closed to an open status. This latching solenoid valve only requires electrical energy to change the open/closed status of the valve and therefore does not require energy to maintain the valve position, unlike the KOGE™ example given above. The lower energy demand of this valve is particularly advantageous to the treatment system 100, 200, 300, 400 described herein, where long durations of valve timing may be applied by the controller. However, the power saving advantage of this solenoid valve also introduces the risk of total vacuum pressure loss to the wound treatment device 3 in the event that power is lost to the vacuum pressure unit 2, which can be mitigated by the inclusion of a capacitor component within the electrical circuit connecting to the solenoid valve.

The air inlet valve 18 does not operate as a pressure relief valve, i.e. the air inlet valve is not controlled to ‘crack open’ to limit a pressure at the wound. The air inlet valve is opened and closed based on a predetermined time period, i.e. the control of the air inlet valve is temporal control, not pressure control, as explained in more detail below.

The fluid inlet valve 22 includes an actuator such as a solenoid in electrical communication with the controller to drive the valve between open and closed positions. The KOGE™ solenoid and Takasago Fluidic System latching solenoid valves described above could be used for this purpose. Both these valves contain moving parts which directly contact the treatment fluid flowing from the treatment fluid reservoir 26 to the wound treatment device 3 via the secondary conduit 12. To be suitable for human use the fluid contacting components would be required to be made from biocompatible materials while also being supplied sterile.

In this case the use of pinch valve or similar non-fluid contacting fluid control valve such as the ASCOR 390NO12330 2-way nominally closed pinch valve is desired. These pinch valves have an open channel or receptacle to receive a tube that is connected to a treatment fluid reservoir 26 such as an IV bag. An example of would be the small tube contained within the Braun Medical® Single Chamber IV Infusion Set, where the pinch valve would replace the roller clamp component within the IV infusion set. In the illustrated embodiment of FIGS. 9 and 10, a solenoid operated pinch valve may be applied to a tube extending between the fluid port 25 and the connection manifold 21. A solenoid operated pinch valve has an internal spring that ensures a ferromagnetic plunger component pinches the tube closed when the tube has been inserted into the valve. The controller provides a supply of electrical current to an internal coil of the solenoid where the resultant magnetic force retracts the plunger against the force provided by the spring into an open position to release the tube, thereby allowing treatment fluid to flow through the tube. The controller continues to supply electrical current to the solenoid when the supply of fluid is required. When the supply of treatment fluid from the reservoir 26 is no longer required the controller ceases to supply electrical current to the solenoid which results in plunger returning to the closed position via the force applied by the internal spring.

### Dressing Control Valve

The dressing control valve 31 includes an actuator such as a solenoid in electrical communication with the controller to drive the valve between open and closed positions. Any suitable actuator, such as the KOGE™ solenoid and Takasago Fluidic System latching solenoid valves described above could be used for this purpose.

### Tubing

FIGS. 31 and 32 present cross sections for two dual lumen conduits for connecting between the vacuum unit 2 and the treatment device 3. The conduit shown in FIG. 31 has a circular outer wall. This conduit is preferred for wounds treatments where the conduit must be subsequently removed without opening the wound, for example internal wound treatment. The round or circular outer wall allows the conduit to be rotated upon removal to gently release tissue adhered to the side of the conduit which can cause discomfort to the patient.

To provide for a circular outer wall, the primary and secondary lumens 11, 12 are provided side-by-side. The secondary or supply lumen 12 is provided with a circular cross section, and the primary or removal lumen 11 is provided with a crescent shaped cross section to partially wrap or around the secondary lumen 12. The primary lumen 11 has a cross sectional area greater than the cross sectional area of the secondary lumen 12.

The conduit cross section of FIG. 32 is preferred for use with or as part of a wound treatment device for treating an external wound, as described below with reference to FIGS. 39 to 42. The conduit comprises a primary or removal lumen 11 and a secondary or supply lumen 12 side-by-side with an internal wall W separating the two lumens, and with the two lumens comprising a circular cross section.

By example, the supply lumen may have a cross sectional area of about 1.7 mm² and the removal lumen may have a cross sectional area of about 9 mm². A typical length of tubing between the vacuum unit and treatment device is around 1000 mm. In other embodiments the cross sectional area of the supply lumen could range from approximately 0.7 mm² to 3 mm², with the primary lumen varying from approximately 2 mm² to approximately 30 mm² with the tube length supplied anywhere from approximately 200 mm in length to approximately 1500 mm in length.

### Wound Treatment Device

An example treatment device 3 for use in internal wounds is shown in FIG. 7, which is similar to the treatment device shown schematically in FIG. 6 comprising a single tubular flow path in the wound treatment site. The treatment device 3 provides a fluid flow path through the wound treatment site 4. The treatment device 3 includes a perforated conduit 3a having an upstream end 3b and a downstream end 3c. Fluid, for example air, is provided to the treatment device and wound site via the upstream end 3b of the treatment device 3. Fluid, for example air and exudate, is removed from the wound and treatment device 3 via the downstream end 3c of the treatment device 3. In the illustrated embodiment, the treatment device 3 provides a single flow path through the treatment device; i.e. the treatment device conduit does not include branches. In such an arrangement, where portions of the treatment device conduit are close together, a ‘short circuit’ path SC can exist, whereby flow occurs preferentially between two portions of the treatment device conduit 3a that are close together. This can prevent fluid flow reaching other areas of the treatment site, preventing flow to and from those areas.

In some embodiments a treatment system may comprise an external wound treatment device to deliver treatment fluids and/or air and provide for the subsequent removal of fluids from a wound whilst maintaining a sub-atmospheric (negative) pressure environment.

FIG. 39 shows a treatment system comprising an external wound treatment device 40. The vacuum unit 2 is connected to a source of therapeutic fluid 26 and a wound exudate reservoir 6 via conduits, and to the treatment device 40 via a dual lumen conduit 5, as described previously for embodiments 200 and 300 of FIGS. 8 to 12. However, the wound treatment system may be without a therapeutic fluid supply, as described for the embodiment 100 of FIGS. 6 and 7.

FIG. 40 is an illustrative cross-sectional view of the external wound treatment device 40 shown in FIG. 39, where the section view corresponds to the line denoted with an ‘x’ in FIG. 39.

The external wound treatment device 40 comprises a porting component or layer 41 (wound filler), a cover dressing or layer 42, a supply conduit 12 and (separate) removal conduit 11. The porting component 41 is shown to be placed within a wound cavity to fill a treatment space of the wound. The cover dressing is adhered to an area of non-compromised intact skin surrounding the wound (also known as the peri-wound). The dressing cover 42 is made from materials that enable adhesion to the peri-wound whilst providing an airtight seal, such as polyurethane film that contains a layer of pressure sensitive acrylic adhesive, to allow the maintenance of vacuum pressure within the wound site. Such materials are known in the art.

The treatment device 40 comprises two separated conduits 11, 12 within the treatment space of the wound. The conduits 11, 12 may be terminal portions of the supply conduit 12 and removal conduit 11 extending between the treatment device 40 and the vacuum unit 2 described earlier. In the example of a dual lumen conduit, a terminal portion of the dual lumen conduit may split into two separate limbs, a removal or exudate limb (the removal conduit) and a supply limb (supply conduit).

As discussed above, the supply conduit 12 provides for the supply of air, or air and fluids, to the wound, including treatment and therapeutic agents and the supply of sterile air. The removal conduit 11 provides for the removal of exudate fluid from the wound.

In FIG. 39, both conduits 11, 12 have perforations or apertures 11a, 12a of equal or differing sizes to permit the exchange of fluid to the target treatment site. Terminal ends of the conduits may be occluded/blanked so that flow out of and into the respective conduits is through perforations along their lengths. Alternatively, terminal ends also present an outlet and inlet for the supply and removal conduits respectively. The perforations are spaced apart along the conduit and may be placed at repeating or varying distances along the conduit to reduce a potential pressure loss that may occur along the length of the conduit. The perforations or apertures 11a, 12a provide an outlet or outlets from the supply conduit 12 and inlet or inlets to the exudate conduit 11.

The porting component or porting layer 41 may be formed from any suitable biocompatible material that can facilitate the flow of fluid through or around the porting layer 41, including but not limited to polyurethane foams, polyvinyl alcohol foams, non-woven fabrics, spacer fabrics, gauzes, reticulated foams, plastic mesh materials or elastomeric components constructed from silicone, thermoplastic elastomer or polyurethane which provide sufficient structural integrity to prevent the collapse of the cover dressing material into the wound under an applied vacuum pressure.

A wound contacting component may also be additionally placed between the wound and the porting component to promote wound healing and may comprise extracellular matrix (ECM) graft materials such as decellularised human or animal tissues isolated from various organs and from a variety of animal connective tissue and basement membrane sources. Other possible wound contacting components include natural polymeric materials such as a protein, polysaccharide, glycoprotein, proteoglycan, or glycosaminoglycan. Examples may include collagen, alginate, chitosan and silk. Alternatively, or additionally, wound contacting components may comprise synthetic polymeric materials such as polypropylene, polytetrafluoroethylene, polysiloxanes (silicone), polyglycolic acid, polylactic acid, poliglecaprone-25, or polyester. The wound contacting component may comprise a multiple layer combination of one or more of the above materials.

The treatment space of the wound shown in FIG. 40 is defined by the cover dressing 42 and the wound treatment surfaces of the external (open) wound. The treatment device 40 is applied to the treatment space of the wound to facilitate the supply of fluids to and the removal of fluids from the treatment space. The treatment space contains the fluid supply conduit 12, exudate fluid removal conduit 11 and the porting component or layer 41.

A fluid flow from the supply conduit 12 through the porting component 41 and out to the exudate fluid removal conduit 11 is indicated by the arrows in FIG. 40. The porting component 41 ensures that supplied therapeutic fluid and/or air is distributed to the treatment surfaces within the wound whilst a level of negative pressure is maintained within the wound treatment space.

As shown in FIGS. 39 and 40, the fluid supply conduit 12 and the exudate or removal conduit 12 are arranged to avoid or prevent a ‘short circuit’ flow path between the two conduits and through the wound treatment space. The arrangement improves fluid flow through a substantial portion of the treatment space and preferably substantially the entire treatment space. A short circuit may result in a preferential flow of fluid to only portions of the treatment space rather than the supply of fluid to substantially the entire treatment space.

To avoid a short circuit path through the wound treatment space, in preferred embodiments, the two conduits 11, 12 are positioned at opposed locations of the porting layer, i.e. at or adjacent perimeter portions of the porting layer and/or the wound treatment site. In FIGS. 39 and 40 the two conduits are placed at or adjacent opposed perimeter portions of the porting layer or wound treatment space. The supply and removal conduits 12, 11 are positioned preferably at a maximum distance apart in the wound treatment space.

In the illustrated embodiment the supply and removal conduits are placed on one side of the porting layer. However, in other embodiments, the conduits may be arranged on opposed sides of the porting layer or may be embedded in the porting layer. When placed on a side surface of the porting layer (for example the outer most surface, the furthermost surface away from the wound), preferably the supply and removal conduits are provided to the porting layer so that the inlet apertures 11a and outlet apertures 12a face towards or are placed against the surface of the porting layer 41.

In FIG. 39, the supply and removal conduits 12, 11 are arranged so that there is a constant distance between the outlets 12a and inlets 11a of the respective conduits. A minimum distance between the inlets 11a and the outlets 12a is many times greater than a maximum distance between adjacent outlets 12a along the length of the supply conduit 12. The minimum distance between the inlets 11a and the outlets 12a is many times greater than a maximum distance between adjacent inlets 11a along the length of the exudate conduit 11. For example, the minimum distance between the inlets and outlets may be 5, 6, 7, 8, 9, 10 times greater than the maximum distance between adjacent inlets and/or adjacent outlets.

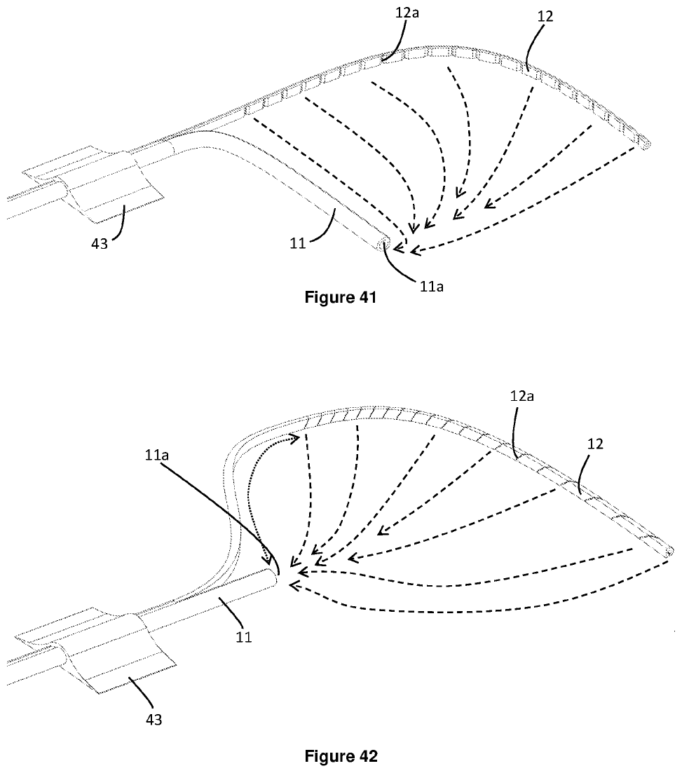

In some embodiments, perforations/apertures 12a may be provided in the supply conduit 12 only, without perforations or apertures along the removal conduit 11. With reference to FIGS. 41 and 42, the supply conduit 12 is provided with outlets 12a along its length as described above, and the removal conduit 11 is without inlets along its length. Fluid flow from the supply conduit 12 to the removal conduit 11 is from the outlets 12a spaced apart along the supply conduit 12 and an open end of the supply conduit 11, and via the porting layer 41 to the end of the removal conduit 11, as indicated by the dashed lines in FIGS. 41 and 42. The open end of the removal conduit 11 provides an inlet aperture 11a.

Alternatively, perforations/apertures 11a may be provided in the removal conduit 11 only, without perforations or apertures along the supply conduit 12, in which case an open end of the removal conduit 12 provides an inlet aperture 12a.

In a preferred embodiment, the treatment device 40 comprises a dual lumen conduit 5 comprising a supply lumen and a removal lumen. An end portion of the conduit 5 is split along its length to separate the conduit into a supply conduit portion 12 comprising the supply lumen and a removal conduit portion 11 comprising the removal lumen. For example, the conduit shown in FIG. 32 is split along its length through the internal wall W separating the two lumens without breaking into either lumen. To create apertures in the side wall of the supply conduit, or the outlet conduit, cuts may be provided along the length of the conduit. For example, as shown in FIG. 41, spaced apart notches are made through the conduit wall to break into the lumen and create an aperture through the wall of the conduit. In the illustrated embodiment the cuts or notches are made through the internal wall W of the conduit. In FIG. 42, a spiral cut is made along the length of the conduit 12 to provide a flow path along the length of the conduit. In some embodiments, as the internal wall W of the dual lumen conduit has been separated between the two limbs, the internal wall can have a thinner section than an outer wall portion of the conduit, such that the spiral cut fully penetrates the internal wall portion of the conduit without fully penetrating the outer wall portion of the conduit, to present spaced apart apertures 12a along the length of the conduit. The embodiments of FIGS. 41 and 42 provide a convenient cost-effective method for providing supply and removal conduits to a wound treatment device and avoids the requirement for connectors. Each notch or cut preferably provides a small aperture though the wall of the conduit, for example for the supply conduit an aperture with a diameter of about 0.6 mm or less.

As shown in FIGS. 41 and 42, the treatment device may include a bridging component 43 through which the dual lumen conduit 5 passes, as described in U.S. provisional patent application 62/568,914, the contents of which is incorporated herein by reference. The bridging component 43 is secured to the patient's skin and facilitates a seal between the top cover 42 of the treatment device and the patient's skin.

### Manifolds

As described with reference to FIGS. 3, 6, 7, 9 and 10, in some embodiments the vacuum unit 2 comprises a connection or interface manifold 20, 21 for connecting the wound treatment device 3, 30, 40 to the vacuum pressure unit 2, and in the embodiments of FIGS. 9 to 12 and FIG. 39, for connecting the treatment fluid reservoir 26 to the fluid supply pathway of the vacuum unit 2.

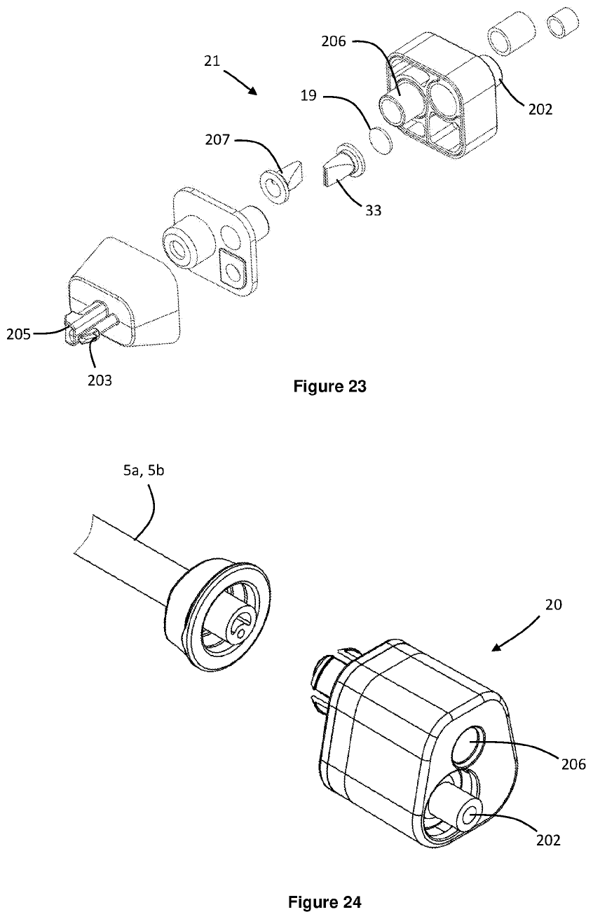

FIGS. 24 to 26 illustrate the interface manifold 20 for use in the embodiment of FIGS. 6 and 7. The manifold 20 comprises a first fluid flow path 201 with a first inlet 202 and first outlet 203, and a second fluid flow path 204 with a second fluid inlet 205 and a second fluid outlet 206. The first inlet 202 connects to the air inlet valve 18 and the first outlet 203 connects to the supply conduit 12 from the vacuum unit 2 to the treatment device 3. The second inlet 205 connects to the removal conduit 11 from the treatment device 3 to the vacuum unit 2, and the second outlet 206 connects to the pump inlet 56. The first outlet 203 and the second inlet 205 provide a fluid outlet to the treatment device 3 and a fluid inlet from the treatment device 3. In the illustrated embodiment the manifold comprises a sterile filter 19 in the first fluid path 201. In this embodiment, the sterile filter is provided as a separate assembly comprising a housing 19a and internal filter element with the filter housing 19a received in the first flow path 201 of the manifold 20 (omitted in FIG. 26). Thus, the connection manifold 20 provides a convenient connection interface between the inputs and outputs of the system with respect to the treatment device 3 while also ensuring a sterile interface for the air inlet to the treatment system 100. The illustrated embodiment also comprises a one-way valve 207 in the second flow path 204 to prevent back flow in the removal conduit from the pump to the treatment device.

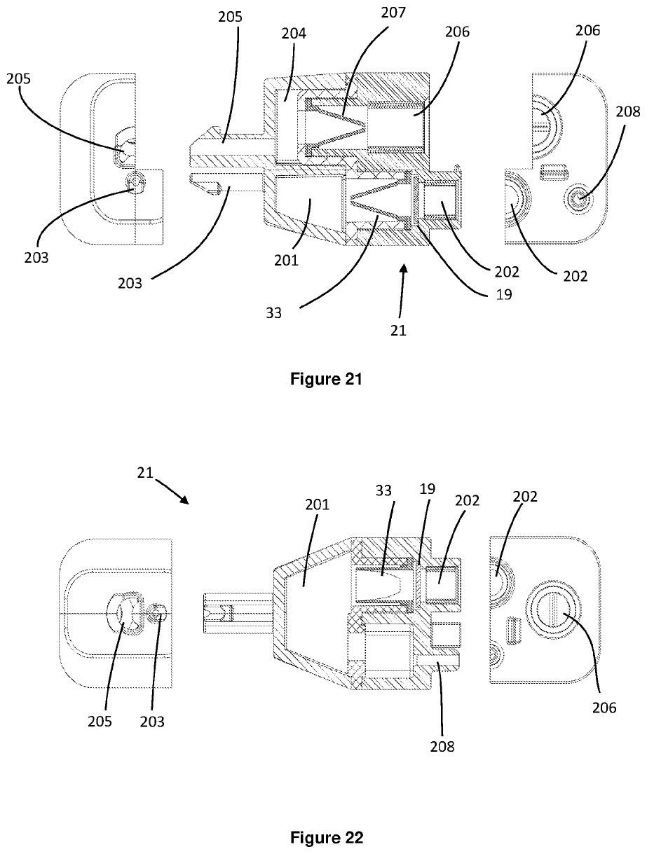

FIGS. 20 to 23 illustrate the interface manifold 21 for use in the embodiments of FIGS. 9 to 12. The manifold 21 comprises a first fluid flow path 201 with an air inlet 202 and a treatment fluid inlet 208 in fluid communication with a first outlet 203, and a second fluid flow path 204 with a second fluid inlet 205 and a second fluid outlet 206. The air inlet 202 connects to the air inlet valve 18 and the treatment fluid inlet 208 connects to the treatment fluid reservoir 26. The first outlet 203 connects to the supply conduit 12 from the vacuum unit 2 to the treatment device 3. The second inlet 205 connects to the removal conduit 11 from the treatment device 3 to the vacuum unit 2, and the second outlet 206 connects to the pump inlet 56. The first outlet 203 and the second inlet 205 provide a fluid outlet to the treatment device 3 and a fluid inlet from the treatment device 3.

In the illustrated embodiment the manifold 21 comprises a sterile filter 19 in the first fluid path. The sterile filter 19 comprises a filter membrane received in the first fluid flow path 201. Thus, the connection manifold 21 provides a convenient connection interface between the inputs and outputs of the system with respect to the treatment device 3 while ensuring a sterile interface for the air inlet to the system 200, 300. In a preferred embodiment the manifold comprises a one-way valve 207 in the second flow path 204 to prevent back flow in the removal conduit from the pump to the treatment device. The connection manifold 21 may comprise an additional one-way valve 33 in the first flow path, positioned adjacent to the sterile filter 19 to prevent fluid ingress from the treatment fluid damaging the sterile filter 19.

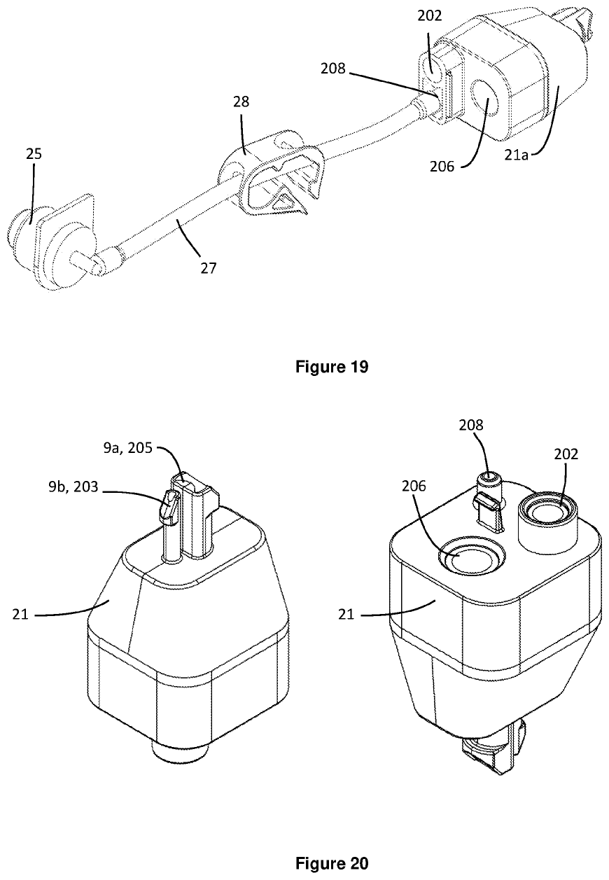

FIG. 19 illustrates a manifold 21a very similar to the manifold 21 of FIGS. 20 to 23. The manifold 21a has the same internal features as manifold 21 described above. The manifold 21a is connected to a connection port 25 via a tube 27 for connecting to the treatment fluid reservoir 26 external to the vacuum unit 2. The manifold 21a (or 21), tube 27 and connection port 25 are preferably provided as a single sterile connection assembly for the pump unit 2. Thus, the connection assembly provides a convenient connection interface between the inputs and outputs of the system with respect to the treatment device 3 while ensuring a sterile interface for the air inlet and the treatment fluid inlet to the system 200, 300.

A tube clamp 28 may be provided to the tube 27 to provide a means to clamp the tube 27 shut once treatment fluid is no longer required. In an alternative embodiment the tube clamp 28 may be partially closed to provide a means to control a flow of treatment fluid into the system, or may be replaced with a roller clamp to provide a means to control a flow of treatment fluid into the system. Additionally, or alternatively, an orifice may be included, e.g. within the tube 27 or at the manifold 21a to enable the Pv sensor to measure the resultant pressure drop across the orifice when the treatment fluid is flowing. The pressure measured at the Pv pressure sensor will allow the flow rate of treatment fluid from the reservoir 26 to be calculated via the application of Bernoulli's equation. In an alternative embodiment the tube clamp 28 may be replaced with an electrically actuated valve in electrical communication with the controller, e.g. valve 22 as previously described with reference to FIGS. 9 and 10. For example, the valve 22 may be a solenoid operated valve, e.g. a solenoid operated pinch type valve, which operates a plunger biased to a closed position by a spring. The controller 17 operates the solenoid to retract the plunger against the spring bias to open the valve. The valve may pinch the tube line 27 shut to create a vacuum tight seal. In some embodiments, as illustrated in FIGS. 11 and 12, the system may be without a treatment fluid valve controlled by the controller.

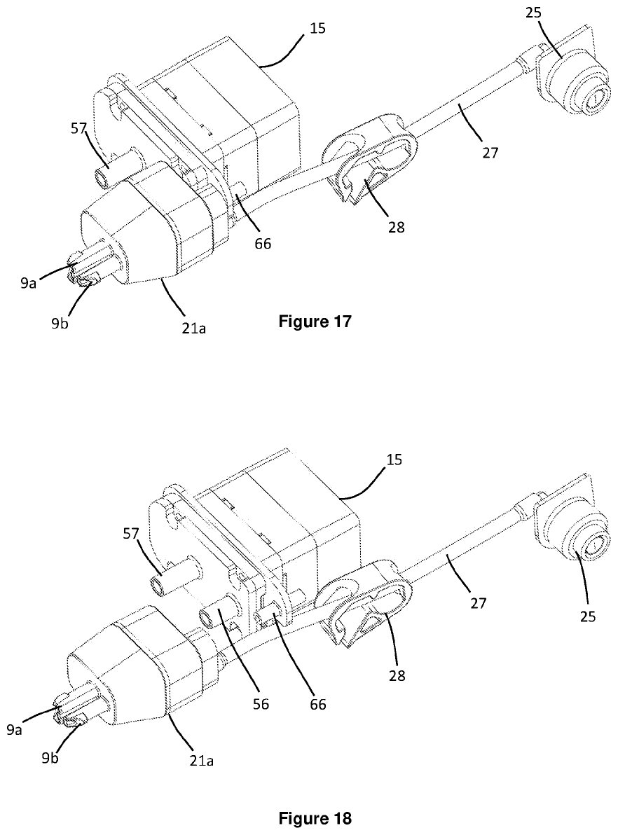

FIGS. 17 and 18 show the manifold 21a of FIG. 19 connected to the pump assembly 15, with the pump inlet 56 connected directly to the second outlet 206 of the second flow path 204 of the manifold, and the air inlet 202 of the manifold connected directly to the through spigot 66 of the pump assembly 15 for connection to the air inlet valve 18.

The manifold one-way valve 207 is preferably a resilient/flexible valve, for example duck bill valves, as described above in relation to the one-way valves incorporated into the pump assembly 15.

The connection manifolds 20, 21, 21a preferably comprise moulded parts welded or otherwise assembled together to form a fluid and air tight assembly. The manifolds 20, 21, 21a provide connectors for fluidly connecting to the other parts of the system, for example spigots or receptacles for being received in or receiving mating tubing/conduits or valve/pump. The valves are preferable molded from liquid silicone rubber.

### General Assembly

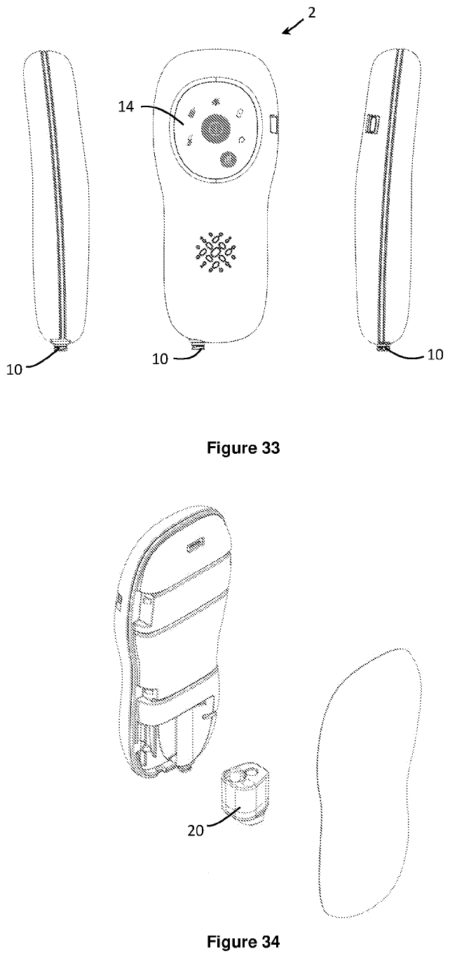

FIGS. 33 to 36 present general assembly drawings for the vacuum unit 7 for the embodiment of FIGS. 6 and 7.

As described earlier, the vacuum unit 2 comprises an enclosure for housing the various components of the unit including the pump assembly 15 with motor 13, user interface 14, battery 16 and controller 17, air inlet valve 18 with actuator, pressure sensors Pv, Pp and sterile filter 19. In this embodiment, the vacuum unit 2 comprise the connection manifold 20 described above with reference to FIGS. 24 to 26. The connector 10 for communication with the collection reservoir 6 is also shown in the Figures, however connecting hoses or tubing/conduits between the manifold 20 and air inlet valve 18, and between the connector 10 and the pump outlet 57, and the conduit from the manifold 20 to the treatment device 3 have been omitted for clarity. The electrical wiring also omitted for clarity.

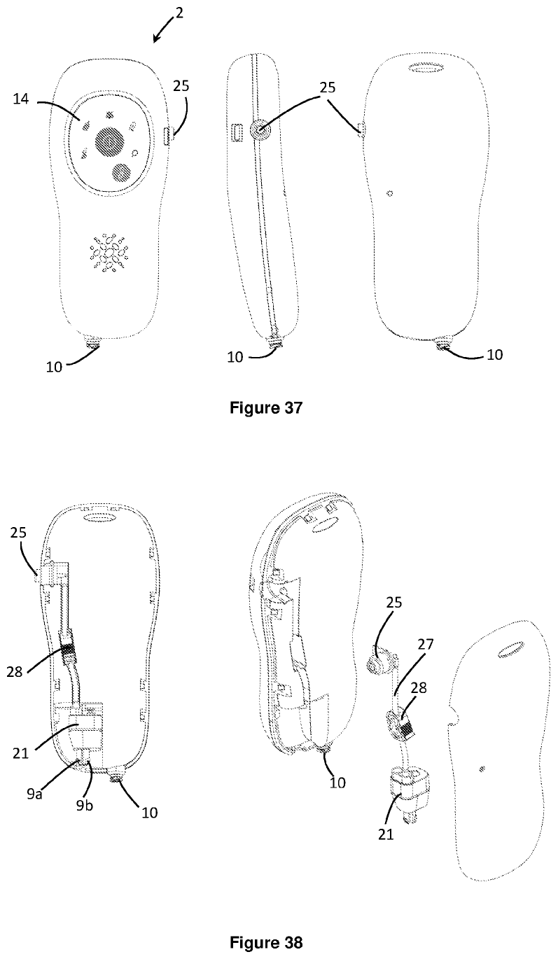

FIGS. 37 and 38 present general assembly drawings for the vacuum unit 2 for the embodiment of FIGS. 11 and 12, additionally including tube clamp 28 on tube 27 connecting the manifold 21/21a to the treatment fluid reservoir 26, as described above with reference to FIG. 19. The illustrated embodiment is similar to the embodiment of FIGS. 33 to 36, however is additionally provided with the alternative interface manifold 21, 21a together with the tube 27, tube clamp 28 and associated connection port 25 for connecting the treatment fluid reservoir 26, with the enclosure incorporating an aperture to receive the connection port 25.

It can be seen from the two embodiments of FIGS. 33 to 38 that the vacuum unit can be easily configured between the two embodiments by choosing the appropriate interface manifold 20, 21 and an enclosure with or without an aperture for the treatment fluid reservoir connection port 25. The controller for each vacuum unit embodiment may comprise a switch to configure it for the ‘air only’ embodiment or the embodiment also comprising the treatment fluid supply option.

### System Operation

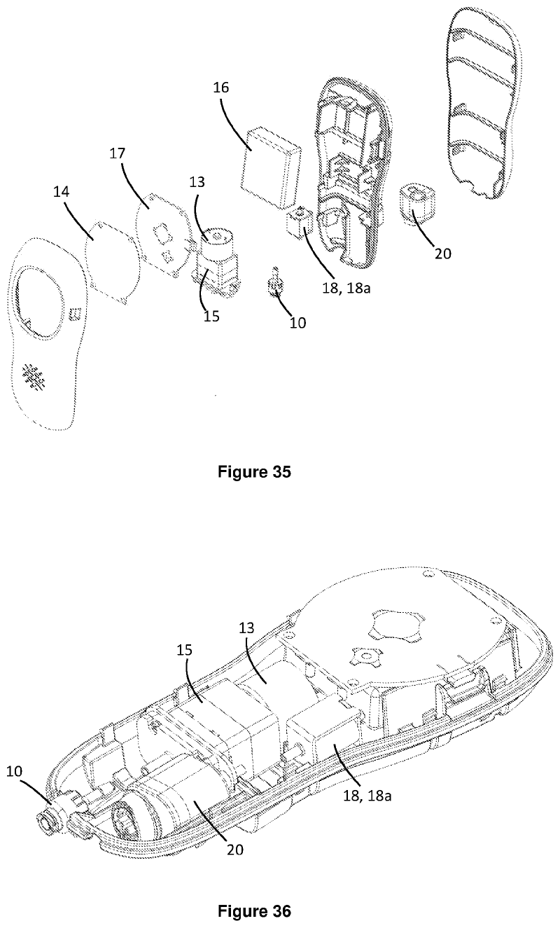

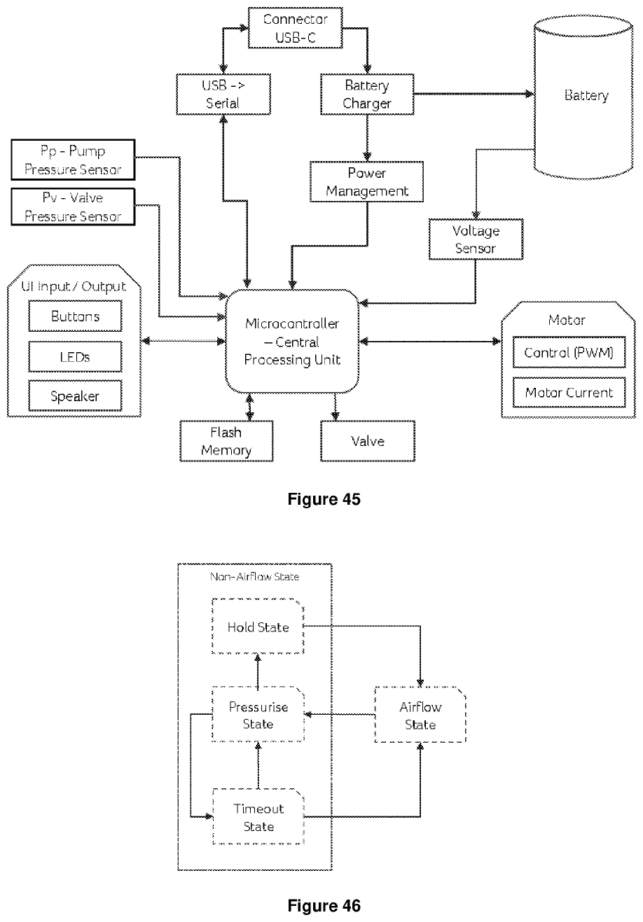

Operation of the treatment system 100 described above with reference to FIGS. 6 and 7 is now described with reference to FIGS. 44 to 50. Initially with reference to FIGS. 44 and 45, the system comprises the user interface 14 to allow a user to operate the system. The user interface may provide visual (e.g. LEDs) and audio indication to the user of system settings and allows inputs, for example one or more buttons 23 (FIG. 56), for example to turn the unit on/off, operate the pump or select operation modes. The controller 17 provides system logic and control algorithms in electrical communication with the air valve actuator (18a in FIGS. 35 and 36), pump motor 13 and pressure sensor(s) Pv, Pp to control the air inlet valve 18 and the pump assembly 15. The controller may also communicate with power management and sensor circuits to manage the power supply 16, for example to provide a battery charge indication to the user via the user interface.

The controller is configured to operate the pump assembly 15 to maintain a negative pressure at the wound 4 via the wound treatment device 3 while opening and closing the air inlet valve. The air inlet valve 18 is opened to introduce air to the wound site while the pump assembly continues to run to maintain a negative pressure at the wound.

Negative pressure treatment can result in a stagnant system, even when the wound continues to produce exudate. In a stagnant system, the system is effectively sealed from the ambient environment and no fluid transfer or flow is achieved from the wound to the exudate reservoir 6. This can exacerbate system blockages due to coagulation of blood, fibrin etc at the wound and/or elsewhere in the system. A blockage ultimately results in failure to provide negative pressure at the wound, defeating the negative pressure treatment.

In order to avoid a stagnated system, the controller opens and closes the air inlet valve 18 while continuing to run the pump assembly 15 to maintain a negative pressure at the wound.

For example, the treatment system 100 is configured to open the air inlet valve 18 to introduce air to the wound site while maintaining a vacuum pressure (a first vacuum pressure) at the wound site 4/wound treatment device 3 of at least 40 mmHg, and preferably at least 50 mmHg. In an example embodiment the treatment system is capable of maintaining vacuum pressure at the wound site/wound treatment device of approximately 50 mmHg to 100 mmHg, or approximately, 60 mmHg to 100 mmHg, or 70 mmHg to 100 mmHg, or 80 mmHg to 100 mmHg, with the air inlet valve open introducing air to the wound. When the controller closes the air inlet valve, the pump continues to operate to maintain negative pressure at the wound. With the air valve closed the vacuum pressure at the wound site 4 may be around 100 mmHg to 150 mmHg (a second vacuum pressure).

Preferably the vacuum pressure maintained at the wound treatment device when the air inlet valve open is at least a substantial portion of the vacuum pressure maintained at the wound when the air inlet valve is closed, or may be equal to the vacuum pressure maintained at the wound when the air inlet valve is closed. For example, the vacuum pressure maintained at the wound with the air valve open may be approximately 30% to 100% of the vacuum pressure maintained at the wound with the air valve closed, or approximately 50% to 100%, or 70% to 100%, or about 80% of the vacuum pressure maintained at the wound with the air valve closed.

With the air inlet valve closed, the vacuum pressure at the wound may be about 20 to 50 mmHg higher than the vacuum pressure at the wound when the air inlet valve is open, or may be equal to the vacuum pressure at the wound when the air inlet valve is open.

In a preferred embodiment the system is configured to cycle the air inlet valve between the open and closed positions while continuing to maintain a negative pressure at the wound. When the air inlet valve is closed the system reverts quickly to a stagnant state. To avoid remaining in a stagnant state that could lead to blockages forming, the controller is configured to again open the air inlet valve while maintaining a negative pressure at the wound, and then again close the air inlet valve. The opening and closing of the air valve continues. The introduce air of into the system while maintaining a negative pressure at the wound promotes movement of fluid from the wound to the reservoir and reduces the risk of blockages. In some embodiments, the treatment system may be configured to continue to open and close the air inlet valve to achieve continuous operation of the pump to maintain fluid flow and avoid remaining in a no-flow or stagnant state for an extended period.

In a preferred embodiment the system is configured so that with the air inlet valve 18 open, the system achieves an equilibrium state, with a flow rate of air into the treatment system through the air inlet valve 18 equal to a flow rate of fluid (e.g. exudate) and air through the pump. In an equilibrium state, the vacuum pressure at the wound treatment device 3 is maintained at or reaches a steady state or constant vacuum pressure level (the first vacuum pressure). The system may achieve the constant vacuum pressure level after a short duration, for example several seconds or less, for example 5 second or less. In some embodiments, with the air valve open and in an equilibrium state, the pressure drop across the treatment device is substantially zero, with substantially all of the pressure drop between the system vacuum pressure and ambient pressure occurring across the inlet restriction, provided for example by the air inlet filter. In some embodiments, with the air inlet valve open and in an equilibrium state, the pressure drop across the treatment device is constant. Introducing air to the wound can create a pressure drop across the wound site—between an upstream side of the treatment device and a downstream side of the treatment device—allowing for the transfer of fluid from the wound 4 to the reservoir 6, to thereby reduce the risk of coagulation and system blockage.

With the air valve closed, the pump is controlled to maintain a negative pressure at the wound and a flow rate from the wound to the pump is proportional to the patient's wound response; i.e. the flow rate is proportional to the exudate produced at the wound. With the air inlet valve closed, the pump is controlled to maintain the vacuum pressure at the wound treatment device at a steady state or constant vacuum pressure level (the second vacuum pressure). Again, the system may achieve the constant vacuum pressure level after a very short duration, for example several seconds or less, for example 5 second or less. As described above, the first vacuum pressure is less than or equal to the second vacuum pressure.

The steady state vacuum pressure at the wound treatment device 3 with the air inlet valve 18 open may be less than the steady state vacuum pressure at the wound treatment device with the air inlet valve closed. However, the vacuum pressure at the wound treatment device 3 with the air inlet valve open is sufficient for effective negative pressure treatment. As described above, the first vacuum pressure is at least a substantial portion of the second vacuum pressure and may be equal to the second vacuum pressure. Thus, the cyclic opening and closing of the air inlet valve while running the pump to continuously achieve a negative treatment pressure not only improves removal of exudate and reduces the risk of system blockages, but also maintains the negative pressure environment at the wound for effective wound treatment.

Cycling the air inlet valve open and closed while maintaining a negative pressure at the wound achieves a reduced fluid density at the wound site by the introduction of air. Often a height differential exists at the wound site, for example when the patient is upright or in a standing position. A height differential at the wound can result in fluid remaining static in a lowermost location in the wound, with flow in only upper portions of the wound. By introducing air across the wound site, air reaching the lowermost portions of the wound can lift fluid from those lowermost portions and improve fluid movement throughout the wound. The introduction of air essentially allows the system to operate much like an air pump to allow lower density fluid to move ‘uphill’ or against gravity. A preferred embodiment treatment device for providing fluid flow to avoid areas of reduced or zero flow in some portions of the wound is described above with reference to FIGS. 39 to 42.

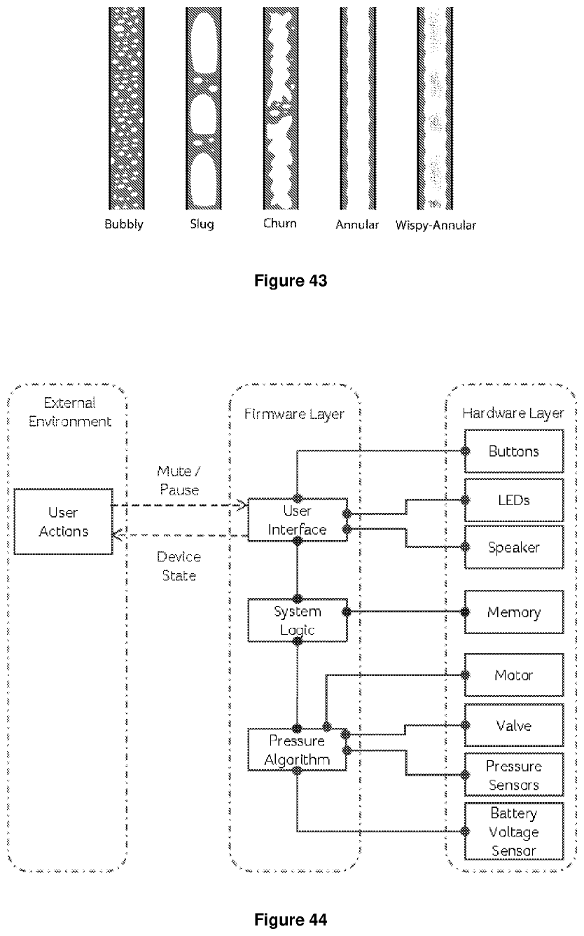

The inventors have additionally identified a preferred mode of operation whereby the air valve is operated between open and closed positions while maintaining a negative pressure at the wound in order to introduce a flow rate of air into the system that achieves a ‘bubbly flow’ or a ‘slug flow’ from the wound site to the reservoir. FIG. 43 illustrates a range of flow types in a fluid comprising both liquid and gas states. Introducing to much air due to leaving the air inlet valve open for too long can result in an annular type flow with exudate flowing along the inner wall of the conduit and air flowing through the middle of the conduit. This can cause the exudate to become stagnant on the wall of the conduit which can lead to the fluid solidifying. A layer of solidified fluid can increase over time leading to a blockage. By cycling the air inlet valve open and closed, liquid exudate can reform a uniform column within the flow path of the system when the air valve is closed, with subsequent opening of the air inlet valve to introduce air results in bubbles or slugs of air passing through the exudate. The air valve is again closed before an annular type flow is achieved. The inventors believe that this results in an improved removal of exudate and reduction in blockages.

An example implementation of cycling the air inlet valve between open and closed during NPT is now described with reference to FIGS. 46 to 50. As illustrated in FIG. 46, the controller is configured to implement an airflow mode or state in which the air inlet valve is opened and the pump is operated to achieve a negative pressure at the wound, and a non-airflow mode or state in which the air inlet valve is closed and the pump is operated to achieve a negative pressure at the wound. In the illustrated embodiment the non-airflow state comprises a pressurise state, a hold state and a timeout state.

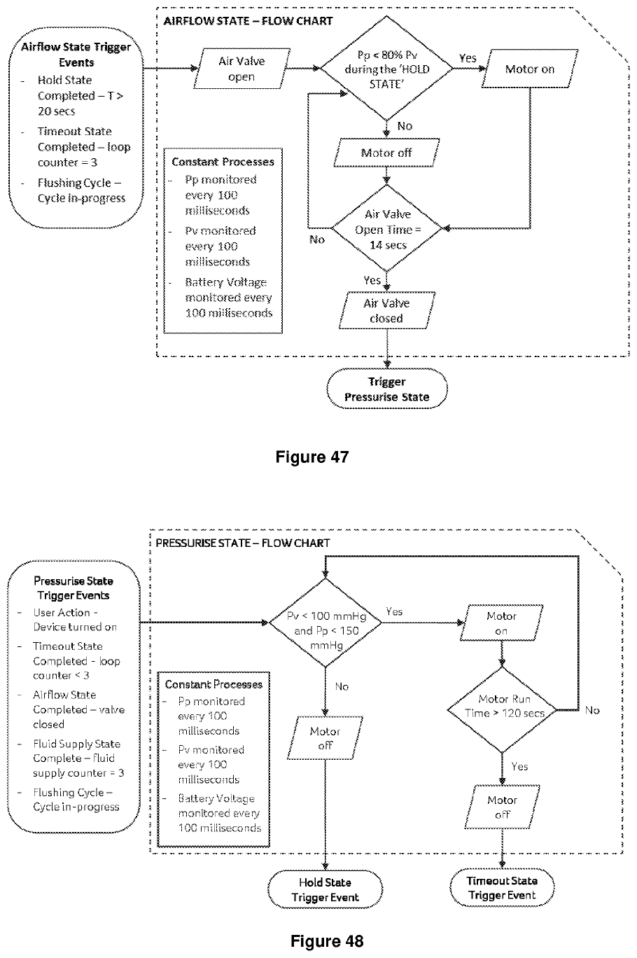

With reference to FIG. 47, in the airflow state the controller opens the air inlet valve to allow air to enter the system on the upstream side of the treatment device and runs the pump to achieve a pressure threshold. For example, if the pressure sensed by the pressure sensor Pp at the downstream side of the treatment device is less than a pressure threshold, the controller runs the pump (turns the pump on). In other words, if the pressure at Pp is greater than or equal to the threshold pressure, the controller turns the pump off.

In the illustrated embodiment, the pressure threshold at the downstream side of the treatment device (Pp) is a portion of a pressure threshold at the upstream side of the treatment device (Pv) when the air inlet valve is closed. In the illustrated embodiment, the pressure threshold at the downstream side of the treatment device (Pp) is 80% of a pressure threshold at the upstream side of the treatment device (Pv) when the air inlet valve is closed. For example, when the air inlet valve is closed, the pressure threshold at the upstream side of the treatment device at Pv is 100 mmHg, and in the airflow state with the air inlet valve open, the pressure threshold at Pp is 80 mmHg.

The pump may repeatedly turn on and off, e.g. under PID control by the controller, to maintain the vacuum pressure at the downstream side of the wound treatment device with the air inlet valve open. Preferably the system is configured to achieve the threshold pressure at the downstream side of the treatment device at Pp in a very short time period, i.e. within several seconds or less, for example 5 second or less. The air inlet valve remains in the open position for a time period. When the air inlet valve is open, the pressure at the wound is maintained constant. In the illustrated embodiment, the air inlet valve remains in the open position for 14 seconds. Once 14 seconds has elapsed, the controller closes the air inlet valve and the controller moves to the pressurise state of the non-airflow state.

The parameters of the above described airflow state are provided by way of example. In some embodiments, the system may be without the pressure sensor Pp on the downstream side of the treatment device. The pump may be provided with a suitable capacity such that the pump is run at a predetermined rate corresponding with a particular system performance to achieve a known or acceptable pressure level at the wound treatment device (the first vacuum pressure) with the air inlet valve open. Additionally, or alternatively, the system may include a pressure relief valve to introduce air to the system at the pump inlet to ensure the vacuum pressure generated by the pump does not increase too high. However, in the preferred embodiment the system includes pressures sensor Pp and the controller operates the pump so that the pressure does not increase beyond a predetermined pressure threshold, being 80 mmHg in the above example. Other pressure thresholds are possible depending on a desired treatment regime. Preferably the controller implements PID control to achieve accurate control of the pump and therefore control of the vacuum pressure at the wound. The controller may use a pulse-width modulation (PWM), or pulse-duration modulation, method in the control of the pump motor.

As shown in FIGS. 6, 7 and 9 to 12 and 57, in the example embodiments the pressure sensor Pv is on an ambient side of the filter. The sterile filter 19 presents a known pressure drop to prevent the vacuum pressure at the treatment device collapsing to ambient pressure when the air inlet valve is open. With the pressure sensor Pv on the ambient side of the filter the sensor Pv essentially measures ambient pressure when the air inlet valve is open. Thus, when the air inlet valve is open, the pressure sensed by sensor Pv is not used in the control of the pump, the pump will run until the pressure sensed by Pp increases above the pressure threshold. In some embodiments, the pressure at Pp will not reach the pressure threshold when the valve is open. The pump may run continuously when the air inlet valve is open, however this is less preferred.

With reference to FIG. 48, in a pressurise state, the air inlet valve is closed, and the controller runs the pump to achieve a pressure threshold to achieve a known or acceptable vacuum pressure at the wound treatment device (the second vacuum pressure). With the air valve closed the vacuum pressure at the wound treatment device may be increased compared to the vacuum pressure achieved in the airflow mode. In the illustrated embodiment, if the pressure sensed by the pressure sensor Pv at the upstream side of the treatment device is less than a 100 mmHg, and the pressure sensed by the pressure sensor Pp at the downstream side of the treatment device is less than 150 mmHg, the controller runs the pump. In other word, if the pressure Pv is greater than or equal to 100 mmHg or pressure Pp is greater than or equal to 150 mmHg, the controller turns the pump off.

The system may be configured to achieve the threshold pressure after a very short duration of closing or opening the air inlet valve, i.e. within several seconds or less, for example 5 second or less. With the air valve closed, since the system is closed or sealed, the system reaches a stagnant or no flow condition very quickly with zero pressure drop across the treatment device and therefore with the pressure at Pv=the pressure at Pp. In the illustrated embodiment, since the pressure threshold at Pv is less than the pressure threshold at Pp, the controller controls the pump based on the upstream pressure sensor Pv, the lower of the two pressure thresholds. However, a pressure drop through the system may occur when tissue debris and/or solidifying materials such as fibrin accumulate within the would treatment device and/or the pump, in which case a pressure differential may develop between the upstream and downstream sides of the treatment device as measured by sensors Pv and Pp. System restrictions may cause the system pressure to reach the higher threshold at the downstream side of the treatment device, before the lower threshold is reached at the upstream side of the treatment device, in which case the pump is controlled based on the downstream pressure sensor Pp to the higher pressure threshold at Pp.

Once the pressure threshold has been reached the controller turns the pump off and moves into a hold state. The pressurise state includes a time-out check so that if the pump has not achieved the pressure threshold (e.g. at Pp) within 120 seconds the motor is turned off and the controller moves to a time out state. This may occur for example due to a blockage within the system or other failure mode, such as a leak.

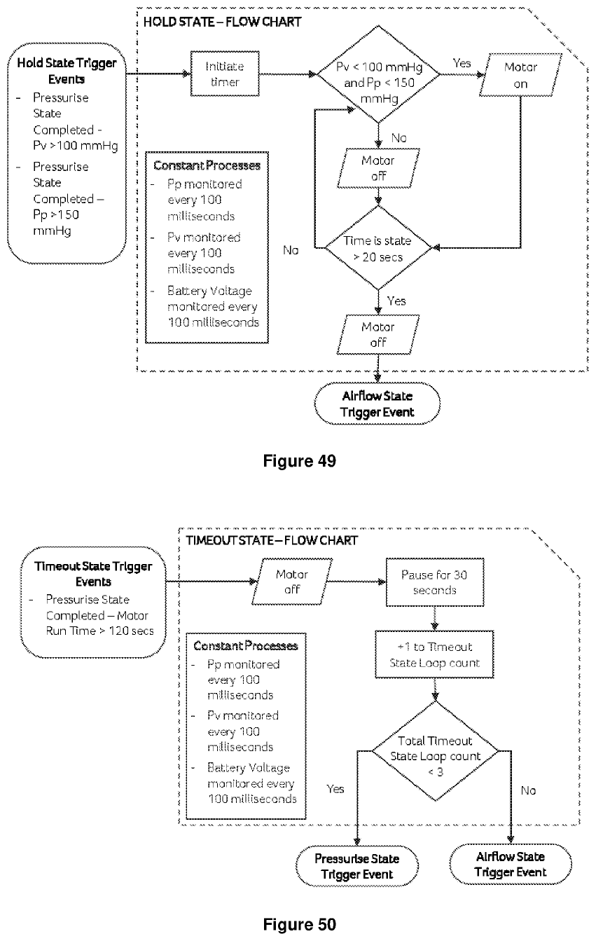

With reference to FIG. 49, in a hold state the controller maintains the air inlet valve in the closed position and continues to operate the pump to maintain the desired or acceptable vacuum pressure at the wound treatment device, by turning the pump on and off, for example under PID control to achieve a desired pressure threshold at Pv or Pp. The controller maintains the vacuum pressure with the air inlet valve shut for a time period. In the illustrated embodiment, the air inlet valve is closed for 20 seconds. Once 20 seconds has elapsed, the controller returns to the air flow mode and the cycling of the opening and closing of the air inlet valve is repeated. The opening and closing of the air inlet valve may be cycled continuously to achieve the above described benefits.

The above example implementation provides an air inlet valve open time of 14 second and an air inlet valve close time of 20 seconds. These time periods are by way of example and alternative time periods may be implemented. However, it is to be noted that the air inlet valve is open for a substantial portion of a total open/close cycle. In this embodiment, the total open/close cycle, or the ‘cycle pitch’ is 34 seconds, with the air inlet valve open for 14 second of the 34 second period, or around 40% of the total cycle. In some embodiments, the air inlet valve is open for at least 10% of the cycle pitch, or at least 20% of the cycle pitch, or at least 30% of the cycle pitch, or at least 40% of the cycle pitch. For example, the air inlet valve open time period may be around the same as the close time period (50% of the cycle pitch). In some embodiments, the air inlet valve may be open for more than 50% of the total cycle.

The above example system configuration provides a cycle time of 34 seconds. However longer or shorter cycle times are possible. As described above, the opening and closing of the air inlet valve required to achieve a slug or bubbly flow from the would site to the reservoir while maintaining negative pressure at the wound is ideal. A maximum valve cycle time may be 1 minute or several minutes. However, the air inlet valve should be open for at least approximately 10 seconds at the above pressures to ensure sufficient air is introduced to the system. The air inlet valve may be open for 10 to 40 second in each air inlet valve open/close cycle.

The time periods for which the air inlet valve is open and closed is dependent on the air inlet flow restriction, the pump capacity, the treatment device configuration and the supply and exudate conduit length and diameter. The above described system components and control parameters are provided by way of example. However, the inventors believe that the system parameters should be selected to enable the air inlet valve to be open for a significant duration while maintaining the negative pressure at the wound at a level useful in the negative pressure treatment of a wound.

With reference to FIG. 50, the example embodiment includes a time out state to safely manage a situation whereby the system is unable to reach an intended negative pressure level. As described above with reference to FIG. 48, if the system is unable to pressurise when the air inlet valve is closed after a predetermined time period (for example 2 minutes) the controller enters the time out state. The controller pauses the pump operation for 30 second and increments a timeout counter. If the time out counter is less than a predetermined count threshold, the controller then returns to the pressurise state to try and pressurise the wound treatment site. If the timeout counter threshold is reached, the controller returns to the air flow state. As described above, introducing air can reduce blockages. The system may have failed to pressurise due to a blockage. Returning to the air flow state may remove a blockage before returning to the pressurise state.

In some embodiments, the treatment system may implement other control parameters not presented in FIGS. 46 to 50. For example, in some embodiments, the system comprises the pressure sensor Pv on the upstream side of the treatment device and the pressure sensor Pp on the downstream side of the treatment device. The controller may operate the pump and/or air inlet valve based on a pressure differential measured between the two pressure sensors. For example, the controller may open the air inlet valve when the pressure differential increases above an upper threshold or is above an upper threshold for a predetermined time period. A system pressure differential may be indicative of a blockage in the system, especially when the air inlet valve is closed. With the air valve closed and with the system in a stagnant state, the pressure on the upstream and downstream sides of the treatment device should be substantially equal. The controller may close the air valve when the pressure differential decreases below a lower threshold or is below a lower threshold for a predetermined time period. The controller may stop the pump and/or the airflow state when the pressure differential increases above an upper or maximum threshold.

As described above with reference to FIGS. 8 to 12, in some embodiments the system is configured to introduce a treatment fluid to the wound. For the system of FIGS. 9 and 10, the controller may be configured to operate the treatment fluid inlet control valve 22 to introduce treatment fluid in a similar way to operation of the air inlet valve 18. The treatment fluid reservoir 26 is preferably at ambient pressure.

The controller opens the fluid inlet valve 22 while operating the pump to maintain a negative pressure at the wound treatment device, to draw treatment fluid into the treatment device. In a preferred embodiment, the system is configured so that with the fluid inlet valve 22 open, the system achieves an equilibrium state, with a flow rate of treatment fluid into the treatment system from the treatment fluid reservoir 26 is equal to a flow rate of fluid (e.g. exudate and treatment fluid) through the pump. In an equilibrium state, the vacuum pressure at the wound treatment device is maintained at or reaches a steady state or constant vacuum pressure level (i.e. a third vacuum pressure). The system may achieve the constant vacuum pressure level after a very short duration, for example several seconds or less, for example 5 second or less. In a preferred embodiment, with the fluid inlet valve open and in an equilibrium state, the pressure across the treatment device is substantially zero.

When the fluid inlet valve is open, the controller may operate the pump to achieve the same pressure at the treatment device that the treatment system achieves when the air inlet valve is open.

With the fluid inlet valve closed, the pump is controlled to maintain a negative pressure at the wound. With the fluid inlet valve closed, the pump may be controlled to maintain the vacuum pressure at the wound treatment device at a steady state or constant vacuum pressure level (a fourth vacuum pressure). Again, the system may achieve the constant vacuum pressure level after a very short duration, for example several seconds or less, for example 5 second or less. When the fluid inlet valve is closed, the controller may operate the pump to achieve the same pressure at the treatment device that the treatment system achieves when the air inlet valve is closed.

The steady state vacuum pressure at the wound treatment device with the fluid inlet valve open may be less than the steady state vacuum pressure at the wound treatment device with the fluid inlet valve closed. However, the vacuum pressure at the wound treatment device with the fluid inlet valve open is sufficient for effective negative pressure treatment. The treatment fluid is not introduced under a positive pressure. Thus, the opening and closing of the fluid inlet valve while running the pump to continuously achieve a negative treatment pressure not only maintains the negative pressure environment at the wound for effective treatment but also provides for the installation of treatment fluid to improve treatment, the removal of exudate, and reduce the risk of system blockages.

The amount of treatment fluid administered to the system can be controlled based on the time the fluid inlet valve is open. A flow restriction (such as a constricting orifice) may be placed between the treatment fluid reservoir 26 and the Pv pressure sensor positioned upstream of the wound treatment device. The resultant pressure drop across this restriction can allow the rate of fluid to be determined from the resulting pressure drop measured by the sensor Pv and the total amount of treatment fluid administered to be calculated. Alternatively, the treatment fluid inlet valve may be open until a differential pressure threshold is achieved or achieved for a time period, or the valve may be opened for a predetermined time period. The treatment fluid inlet valve is preferably opened when the air inlet valve is closed.

With reference to the embodiment of FIGS. 11 and 12, the system is without a treatment fluid inlet valve controlled by the controller. The system administers treatment fluid during negative treatment pressure since the vacuum pressure at the wound draws the ambient treatment fluid into the system. When the air valve is open, air flows to the treatment device, and the flow of air into the system tends to stop the flow of fluid from the treatment fluid reservoir due to the much lower density of the air compared to the density of the treatment fluid. When the air inlet valve is closed, the negative pressure at the wound draws fluid from the treatment fluid reservoir into the system and flood into the wound. Treatment fluid passes through the treatment device and wound and through the pump to the reservoir as the pump maintains a vacuum pressure at the wound. Reopening the air valve again stops the flow of treatment fluid and causes a pressure differential to move fluid comprising treatment fluid and exudate from the wound. Thus, cycling the air inlet valve can also achieve addition and removal of treatment fluid to and from the wound in a cyclic manner. The amount of treatment fluid added is dependent on how long or how much air has been introduced. The amount of treatment fluid introduced to the system may be proportional to an amount of air introduced to the system.

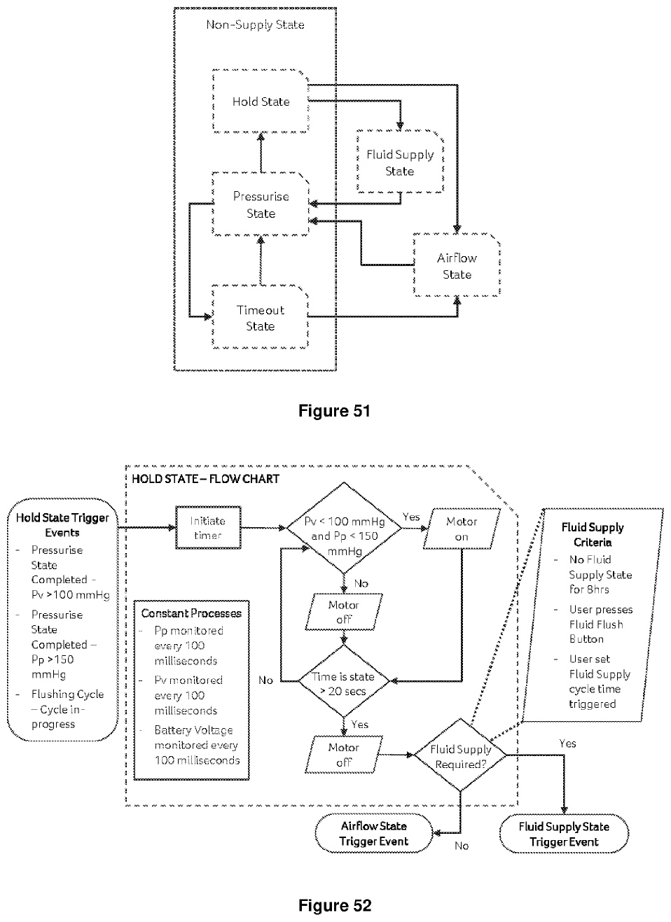

An example implementation of the system of FIGS. 9 and 10 is now described with reference to FIGS. 51 to 54. As illustrated in FIG. 51, the controller is configured to implement a fluid supply mode or state in addition to the airflow state described above. The controller implements a non-supply/non-airflow mode in which the air inlet valve and the treatment fluid valve are closed and the pump is operated to achieve a negative pressure at the wound. In the illustrated embodiment the non-airflow state comprises a pressurise state, a hold state and a timeout state.

The air flow state and pressurise state of FIG. 51 are as described above with reference to FIGS. 47 and 48. Once the airflow state and pressurise state of FIGS. 47 and 48 have been run the controller implements the fluid supply hold state of FIG. 52.

With reference to FIG. 52, in the hold state the controller maintains the air inlet valve in the closed position and continues to operate the pump to maintain the desired or acceptable vacuum pressure at the wound treatment device, by turning the pump on and off, for example under PID control to achieve a desired pressure threshold (at Pp and/or Pv). The controller maintains the vacuum pressure with the air inlet valve shut for a time period, e.g. 20 seconds. Once 20 seconds has elapsed, the controller turns the pump off and checks to see if the fluid supply state is required. If the fluid supply state is not required, the controller returns to the air flow mode and the cycling of the opening and closing of the air inlet valve is repeated as described above with reference to FIG. 46. The controller implements the fluid supply state if no treatment fluid supply has been provided for a predetermined time period, for example 8 hours, or a user set fluid supply cycle time is triggered, or if a user manually requests a fluid supply, for example by pressing a button on the user interface of the vacuum unit.

The time period between activating the fluid supply state is much greater than the air inlet valve open and close cycle time period. For example, the air inlet valve cycle time period may be less than 1 minute and the time period between fluid supply states may be more than 1 hour.

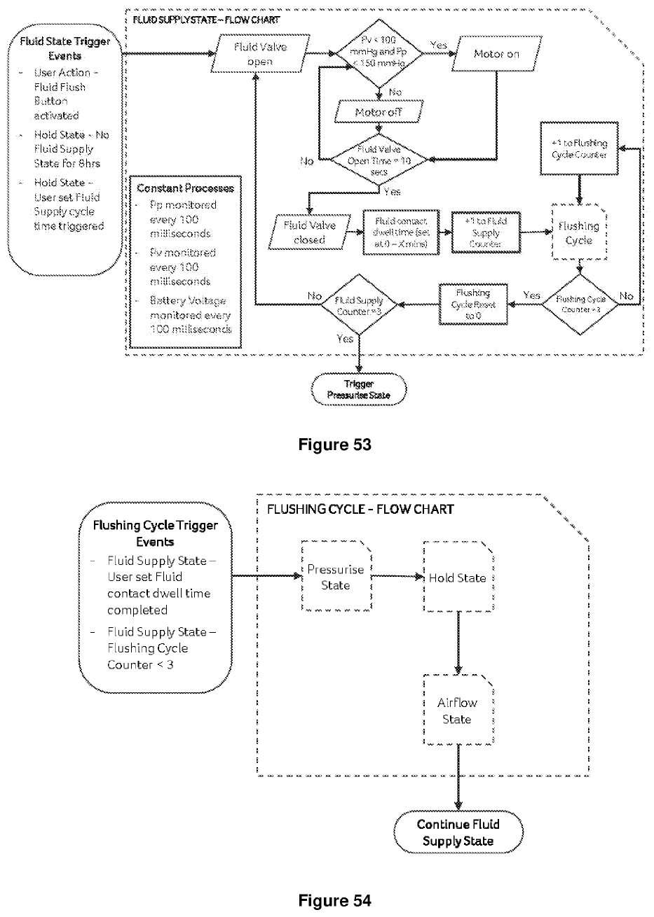

With reference to FIG. 53, in the fluid supply state the controller opens the fluid valve to allow the treatment fluid to flow from the treatment fluid reservoir to the upstream side of the treatment device and runs the pump to achieve a pressure threshold. If the pressure sensed by the pressure sensor Pv at the upstream side of the treatment device is less than 100 mmHg, and the pressure sensed by the pressure sensor Pp at the downstream side of the treatment device is less than 150 mmHg, the controller runs the pump. The control of the pump when the treatment fluid valve is open may be the same or similar to the pump control when the air inlet valve is open as described above. In the illustrated example the controller maintains the fluid valve open for 10 seconds, however other time periods are possible. The controller closes the fluid valve and may allow for a fluid contact dwell time to allow the fluid introduced to the wound to flood or remain in the wound site for a set period of time. The controller may allow for a user input to set the dwell time of between 0 minutes to 10 minutes or other time period. Following the delay to allow fluid contact within the wound the controller enters a flushing cycle to flush the treatment fluid from the wound. In the illustrated embodiment the controller repeats the flushing cycle three times, however the controller may perform the flushing cycle once, twice or more than three times. In the illustrated embodiment the controller repeats the fluid supply state three times before returning to the pressurise state, however the controller may perform the fluid supply state once, twice or more than three times.

With reference to FIG. 54, in the flushing cycle the controller steps through the pressurise state, hold state and airflow state as described above with reference to FIGS. 48 and 49, before continuing with the fluid supply state to repeat the fluid supply state to open the fluid valve again if required as shown in FIG. 53. At the conclusion of the fluid supply state the controller returns to the pressurise state of FIG. 48. The system continues to pressurise, hold pressure and cycle the air inlet valve open and closed as described above.

In the illustrated embodiment, the fluid inlet valve is open for 10 seconds and closed for 102 seconds in each open and close cycle of the fluid inlet valve. The close time is dependent on the dwell time and the combined flushing cycle run time. In the illustrated embodiment, the fluid supply state includes three flushing cycles. With each flushing cycle requiring 34 seconds, and for an example dwell time of zero, in the illustrated example the fluid supply valve is closed for a total of 102 seconds. In the illustrated example the fluid inlet valve is open for around 10% of the cycle pitch. The fluid inlet valve may be open for at least 5% of the cycle pitch, or at least 10% of the cycle pitch, or at least 20% of the cycle pitch.

The fluid supply and flushing states provides a treatment fluid to the wound while maintaining a negative pressure and flushes the treatment fluid from the wound using the introduction of air to remove the fluid and exudate from the wound. As described above, a number of treatment fluid flushes may be provided. This procedure reduces stagnated fluid in the wound, thereby reducing blockages in the system and ensure negative pressure to be continually applied to the wound site.

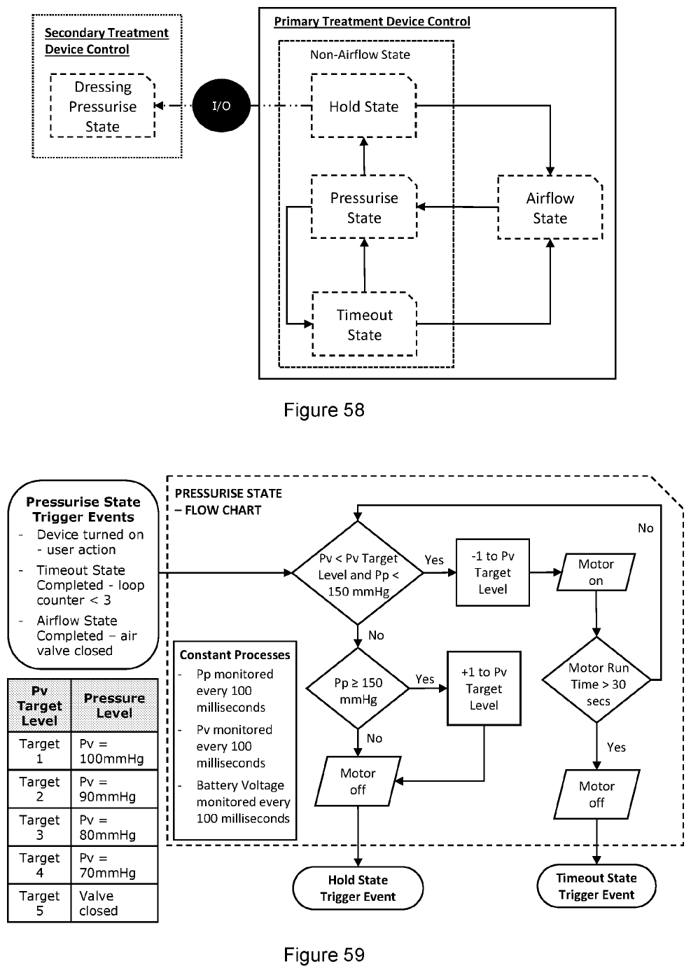

An exemplary implementation of the system 400 of FIGS. 56 and 57 will now be described with reference to FIGS. 58 to 63. The system 400 comprises a first connection via an interfacing sterile manifold connector 20 and connected conduit 5 to an implanted wound treatment device 3 positioned within an internal wound treatment site 4; and a second connection to an external wound treatment device 30 for position over a closed surgical incision 4a. Suitable external wound treatment devices 30 may include those well known in the art, which are configured to apply a negative pressure topically, across the wound 4a. This topically applied negative pressure may act to ‘offload’ the fixation along primary the incision, for example the fixation provided by various mechanical means such as sutures, staples, and/or strips. The external wound treatment device 30 is fluidly coupled to the vacuum unit 2 by a dressing port 31 via a conduit 32.

The operation of the system 400 is via the user interface 14, which enables a user to selectively operate the system. The user interface may provide visual (e.g. LEDs) and/or audio indication to the user to communicate system settings. As illustrated in FIG. 56, in system 400, the user interface 14 includes several buttons 23 to initiate or cease the delivery of negative pressure to the connected external wound treatment device 30, turn the unit power on or off, silence the audible alarm and/or connect the device to a remote wireless receiving device to transmit data regarding the operation or status of the system.

The controller 17 provides system logic and control algorithms in electrical communication with the actuator for the air valve 18, the actuator for the dressing control valve 29, the motor of the pump 15, and pressure sensors Pv, Pp, Pd. The controller 17 is configured to control the air inlet valve 18, dressing control valve 29, and the pump assembly 15 based on the readings at the pressure sensors Pv, Pp, Pd. The controller may also communicate with power management and sensor circuits to manage the power supply 16 or provide battery level warning alarm.

The controller 17 is configured to operate the pump assembly 15 to maintain a negative pressure at the internal wound 4 via the implanted wound treatment device 3 while opening and closing the air inlet valve 18. The air inlet valve 18 is opened to introduce air to the wound site while the pump assembly continues to run to maintain a negative pressure at the wound as described elsewhere within this specification. Additionally, the controller 17 is configured to open the dressing control valve 29 to port negative pressure generated by the pump assembly 15 to the fluidly connected external wound treatment device 30 positioned over the external wound 4a.

As described herein in relation to other system embodiments, negative pressure treatment can result in a stagnant system that can exacerbate system blockages due to coagulation of blood, fibrin etc at the wound and/or elsewhere in the system. A blockage ultimately results in failure to provide negative pressure at the wound, reducing the effectiveness of the negative pressure treatment.

As illustrated in FIG. 58 the controller 17 for the system 400 comprises a first control system for the primary implanted wound treatment device 3, and a secondary control system for the secondary external wound treatment device 30.

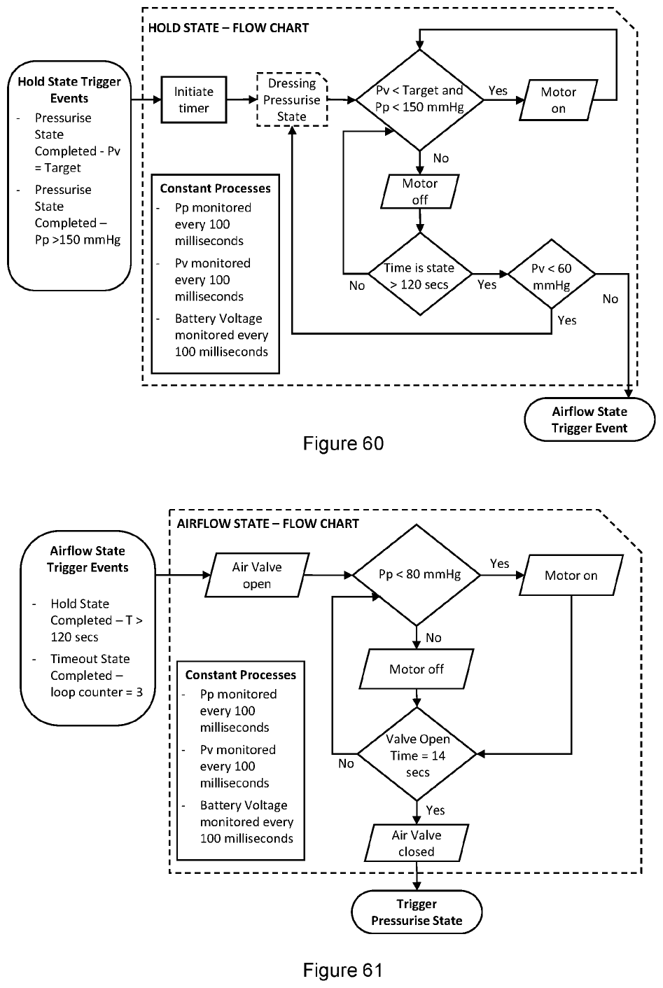

In an illustrative embodiment of the system 400, the controller 17 is configured to operate the pump assembly 15 to achieve a 100 mmHg vacuum pressure level at the valve pressure sensor Pv in the pressurise state when the system is first turned on. That vacuum pressure level is also referred to as the ‘Target 1’ pressure level at the Pv pressure sensor (see FIG. 59). Once the system 400 reaches the target vacuum pressure, the system transitions to the hold state illustrated in FIG. 60. In the hold state, negative pressure may be continued to be applied to the external wound treatment device 30, depending on the operating mode specified by a user.

With reference to FIG. 63, the dressing pressurise state is configured to operate the dressing valve 29 to ensure a vacuum pressure of between 70 mmHg and 95 mmHg is supplied to the external wound site 4a, as measured by the dressing pressure sensor (Pd) between the dressing control valve 29 and the dressing connection port 31. This state continues to operate concurrently while the primary wound treatment device control is in the hold state.

As illustrated in FIG. 60, the hold state is configured to maintain the internal wound treatment device 3 at the primary target pressure, in this example the target of 100 mmHg, when measured at Pv, with a maximum pressure of 150 mmHg being supplied at the pump pressure sensor Pp. The system 400 is held at the hold state for a predefined period, in this example, a duration of 120 seconds. After the predefined period, the system advances to the airflow state illustrated in FIG. 61, unless the Pv vacuum pressure level is below 60 mmHg.

The airflow illustrated in FIG. 61 is similar to the process illustrated in FIG. 47. In this example, the controller is configured such that the pump pressure Pp is targeting a vacuum pressure level of 80 mmHg when the air inlet valve is open. As for the other above described embodiments, the air valve is configured to be held open for 14 seconds after which the air inlet valve is closed as the system transitions to the pressurise state. Also, as described in relation to other embodiments, the air inlet valve may be open for 10 to 40 seconds in each air inlet valve open/close cycle, or may otherwise be varying in durations of open time or configured to detect the equivalent length of conduit that is connected to the device.

In this embodiment the controller is configured to adapt to anticipated changes that can occur system in response to the changes occurring at the wound treatment site 4 and implanted treatment device 3. As the primary treatment device is subjected to repeated cycles through the pressurise, hold and airflow state it has been discovered that a pressure differential between the Pv and Pp pressure sensors can occur in response to changes in the treatment site 4 and/or implanted wound treatment device 3 as a result of tissue in-growth, accumulation of wound debris and many other factors.

In response to these dynamic changes the system adjusts the target pressure level being applied at the Pv pressure sensor during the pressure site to compensate for the changes in the treatment device 3. For example, if the motor has stopped as a result of the Pp pressure sensor being above 150 mmHg the system will drop the target vacuum pressure level from the Target 1 (100 mmHg) pressure being applied at the Pv pressure sensor by a factor of 10 mmHg to a Target 2 pressure of 90 mmHg before advancing to the hold state. If the pressure drop across the implanted treatment device 3 increases again the system will continue to drop the target level by one integer until the Pv pressure level reaches a pressure below 60 mmHg (Target 5). Once the pressure level measured at the Pv pressure sensor reaches this level the system will halt the transition from the hold state to the airflow state which will revert the system to a continuous vacuum pressure level system.

If the vacuum pressure level at Pv returns to 90 mmHg (Target 2), following a drop to below 60 mmHg (Target 5) during the hold state, the system will resume the advancement to the airflow state where the cycling between hold, airflow and pressurise will resume.

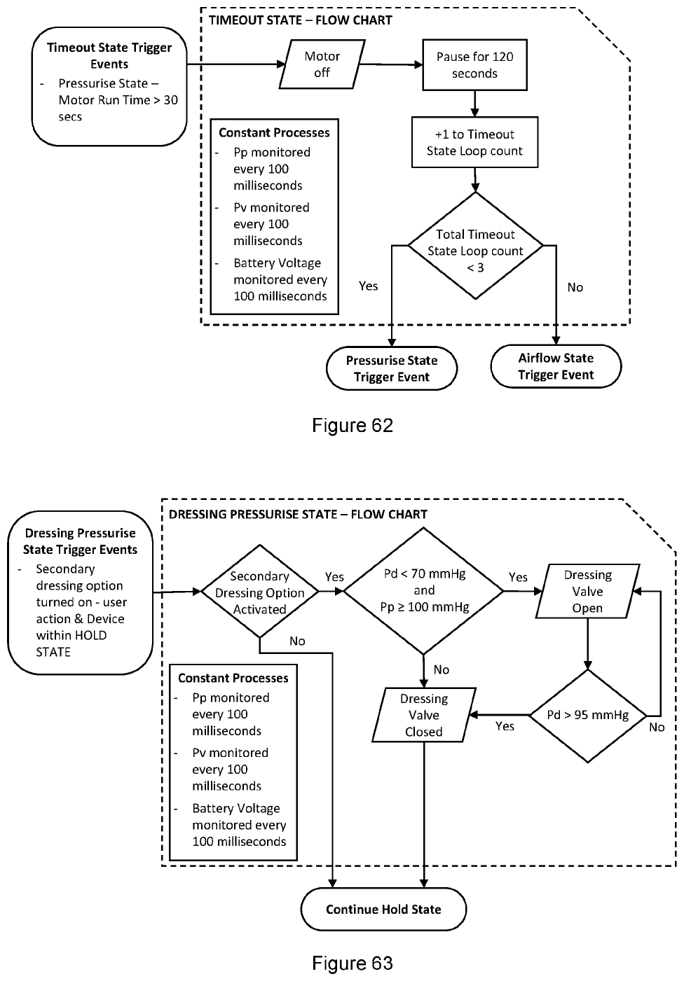

The timeout state as described within FIG. 62 is largely similar to that of FIG. 50 except the system is paused for 120 seconds before the state is advanced towards either the pressurised or airflow state.

### System Specific Operation for an External Wound Treatment Device

Referring again to FIGS. 9 to 12 and 39, in which the vacuum unit 2 is connected to a source of therapeutic fluid 26 and a wound exudate reservoir 6 via respective conduits, and to a treatment device 3 via a dual lumen conduit 5. In some embodiments, the wound treatment device 3 may be an external wound dressing 40 (FIG. 39), and the wound treatment system 200, 300 may not include a therapeutic fluid supply 6 (as also described for the embodiment 100 of FIGS. 2, 3, 6 and 7).

Such systems may be configured to periodically open the air inlet valve 18 to introduce filtered air into the external wound treatment device to achieve a first vacuum pressure level in the absence of a therapeutic fluid supply. The user interface 14 of the vacuum unit 2 may be optionally configured to provide an adjustment means, such as a button and/or other suitable user input, and a corresponding indicator such as a graphical scale and/or LED indicator light that allows the user to adjust the air inlet valve open time to compensate for the level of exudate produced for any given wound and corresponding dressing size.

Exudate produced and therefore, opening times for the air inlet valve 18 may vary depending on the wound size, type, or healing progress. For example, a small wound requiring a 10 cm×10 cm dressing to cover the wound area with a low amount of exudate may be expected to produce approximately 30 mL of wound exudate in a day¹.

In one example embodiment system a 100 cm length of dual lumen conduit 5 with a supply conduit 12 having an internal diameter of 1/16″ (Ø1.6 mm ID) and a removal conduit 11 having an internal diameter of 3/16″ (Ø4.8 mm) will contribute 20 cm³ of volume to the total free volume occupied by the system. The total free volume is defined as the volume occupied by the internal conduits and the volume occupied by the wound treatment device 40. If a low profile non-adherent dressing system, such as those disclosed in the applicant's application U.S. application No. 63/280,787, is applied to the wound with a total dressing height of 5 mm, the volume occupied by the porting layer 41 of the treatment device 40 will be approximately 50 millilitres (50 mL), yielding a total system volume of 70 mLs for this example.

In one embodiment, the vacuum unit 2 of is configured to supply the pump assembly 15 with 3.3V. This yields a 178 mL/min free flow rate of air, and an air inlet valve cycle time of at least 23.6 seconds is required to supply the required 70 mL or 70 cm³ volume of filtered air calculated for the example above, to displace the fluid from the system during a single cycle through the Airflow state.

In an alternative embodiment dressing utilising an open cell reticulated polyurethane foam component (such as Granufoam®) for the porting layer to treat the 10 cm×10 cm wound described above, the volume occupied by the porting layer of the treatment device will be approximately 98.7 cm3 for the same size wound (comprised of 78.7 cm³ of foam+20 cm³ of conduit). Granufoam™ PU foam material has been found to contract from 100 mm×104 mm×25 mm to 82 mm×96 mm×10 mm when the wound treatment space is subjected to −150 mmHg of vacuum pressure. This would require a valve open time of approximately 33.3 seconds (˜about 10 seconds longer).

In a further example, a larger wound requiring a 25 cm×25 cm dressing to cover the wound and with a high amount of exudate may be expected to produce approximately 1,750 mL in a day¹. If the same vacuum unit 2 of the embodiment system and wound treatment apparatus as described above is applied to the wound, a total system volume of 332.5 mL requires an air inlet valve cycle time of at least 112 seconds to supply the 332 cm³ (332 mL) volume of filtered air required to displace the fluid from the during a single cycle through the Airflow state.

In this example it may also be advantageous to provide a user interface 14 that provides the user with an option to increase the frequency airflow cycles in a given day to manage the high level of exudate in the wound, where this example would require at least 6 cycles within a 24 hour period to cope with the 1,750 millilitres of exudate produced.

In some systems in which the primary dressing is an external wound dressing, the vacuum unit 2 may be further connected to a source of therapeutic fluid 26 as described previously for embodiments 200 and 300 of FIGS. 8 to 12. In such embodiments, the user interface 14 may provide input means to enable a user to adjust the dispensed volume of fluid to compensate for the total system volume of the wound treatment system 40.

In one such embodiment, the user interface 14 of the vacuum unit 2 could provide a means for setting the volume of the dressing in a separate adjustment to that of the level of exudate produced at the wound. The user interface 14 may include a button that allows a user to set the free volume of the system, for example, by pressing and holding a button to draw fluid through the system at a set vacuum pressure level, such as 30 mmHg. The set vacuum pressure level for introducing and holding fluid within the system could be set anywhere from 10 mmHg to 200 mmHg, but most preferably is between 10 mmHg and 125 mmHg.

The user interface 14 of the vacuum unit 2 may additionally provide a means to adjust the dwell time for any instilled fluid to be held within the treatment device 40. The hold time may be specified as any time period but most preferably is for a duration of between 1 minute to 30 minutes. The pump unit 2 may additionally include a means to oscillate the vacuum pressure level from the first fluid instillation pressure level to a second pressure level, including the duration of time spent at a first pressure level and second pressure level.

Other variables that may provide useful to adjust via the user interface 14 of the vacuum unit include the operating mode of the pump switching between an oscillating pressure mode to a continuous supplied vacuum pressure mode, or adjusting the time elapsed at each vacuum pressure level.

Other variables that may provide useful for adjustment will be known to those trained in the art.

### Example Embodiment

The effectiveness of a treatment system to remove fluid from the wound according to the present invention is illustrated by an example system setup now described.

The pump described above (with reference to FIGS. 13 to 16) comprising two chambers was connected to a 1 L vessel. The pump was driven by a 12V DC motor at a maximum current of 0.25 Amp to apply a vacuum pressure to the vessel and the vacuum pressure in the vessel was measured to obtain fluid and pressure related properties for the pump.

The pump characteristics from this testing are summarised in the Table 1 below:

TABLE 1
Pump characteristics

| | | Average | Average | | |
|---|---|---|---|---|---|
| | | pressurisation | pressurisation | | |
| | | rate from | rate from | | |
| | | 40 mmHg to | 40 mmHg to | | |
| Drive | | 100 mmHg- | 100 mmHg- | | |
| motor | Approximate | vessel filled | vessel filled | Flow rate of | Flow rate of |
| Voltage | no load rpm | with water | with air | water | air |
| 3.3 V | 1100 | 0.9 mmHg/s | 0.7 mmHg/s | 110 mL/min | 178 mL/min |
| 6 V | 1900 | 1.4 mmHg/s | 1.3 mmHg/s | 220 mL/min | 347 mL/min |
| 9 V | 2800 | 1.6 mmHg/s | 1.8 mmHg/s | 330 mL/min | 519 mL/min |

### System Components:

0.22 micron Filter with 58 mm² filtration area (Steriltech part no. PT021350)
Air inlet valve-Mini Solenoid Valve (KOGE part no. KSV2WM-5A)—rated voltage=4.5 VDC, max current 225 mA
Wound treatment device effective internal diameter=Ø3.7 mm
Wound treatment device effective tube length=470 mm
Wound treatment device internal volume=5.1 mL
Wound treatment device tube perforations=two parallel rows of perforations arranged along the effective tube length, 1.5 mm between adjacent perforations, and 2 mm between the two parallel rows, each perforation 0.5 mm diameter±0.2 mm
Removal conduit effective internal diameter=Ø3.4 mm
Removal conduit length=1000 mm
Removal conduit internal volume=9.1 mL
Air supply conduit internal diameter Ø1.45 mm
Air supply conduit length=1000 mm.

Air supply conduit internal volume=1.7 mL
Total system volume (treatment device volume, removal conduit volume and supply conduit volume)=16 mL.
Conduit between pump and exudate collection reservoir ID=Ø3.2 mm
Conduit between pump and exudate collection reservoir length=300 mm.
Reservoir vents=eight 0.45 micron each with ˜8 mm diameter passages.

The above system components were set up according to the system configuration of FIGS. 6 and 7, with the wound treatment device provided in a flexible bag containing 20 ml of fluid to represent a wound treatment space. The system ran for 3 cycles of opening and closing the air inlet valve. Three cycles were repeated at different air inlet valve open and close cycle times.

In the airflow state with the air inlet valve open, a pressure of 50 mm to 90 mmHg was maintained at the treatment device, with a pump pressure of 80 to 90 mmHg. In the hold state with the air inlet valve closed, a pressure of 100 mmHg was maintained at the treatment device and the pump.

The amount of fluid remaining in the vessel was measured after three cycles of opening and closing the air inlet valve. The system was then left to run for 15 minutes with the air inlet valve continuing to be cycled open and closed, and the fluid remaining in the vessel was again measured. Results from the test are presented in Table 2 below.

TABLE 2
Test results

| | | Percentage | | Fluid |
|---|---|---|---|---|
| Airflow time | Hold Time | of cycle air | | Remaining |
| (air valve | (air valve | inlet valve | Fluid Remaining | after 15 mins |
| open) [s] | closed [s] | open | after 3 Cycles [g] | [g] |
| 2 | 120 | 1.6% | 5.1 | 3.6 |
| 2 | 10 | 17% | 5.1 | 1.5 |
| 14 | 10 | 58% | 2.2 | .2 |

A significant benefit of the system illustrated by the test is the effective removal rate of substantially all fluid from the system by cycling the air inlet valve open and closed and with the air inlet valve open for a significant portion of the cycle time. In this test, the effective removal of fluid was greatest when the air inlet valve was open for a significant portion (58%) of the cycle time period. Further testing indicated that further increases in the air inlet valve open time did not result in further improvements in the effectiveness of the system to remove fluid.

It is hypothesised that an important factor in the effectiveness of the system to remove fluid from the wound is a ratio of the volume of air introduced to the system in each air inlet valve cycle to the volume of the system, while continuing to cycle the air inlet valve open and closed and maintaining the vacuum pressure at the wound at effective negative pressure treatment levels. The volume of air delivered through the system in each valve cycle should be at least a substantial portion of the volume of the treatment system. The volume of the treatment system is defined as the combined internal volume of the supply conduit, the treatment device, and the return conduit, e.g. the volume of the system from the inlet restriction (the inlet filter) to the pump inlet.

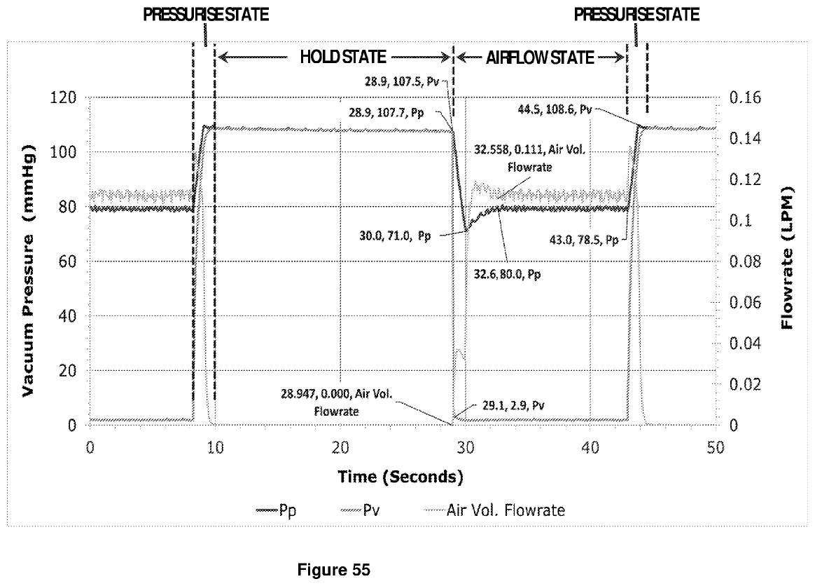

To determine the volume of air added to the system during an inlet valve cycle, the same test set up described above was used but with an inlet filter area of 12.5 mm² and a 4.8 mm diameter 1.5 m length tube representing the supply conduit, treatment device and return conduit, presenting a system volume of 27 mL. The performance of the system is illustrated by the chart presented in FIG. 55.

With reference to FIG. 55, with the air inlet valve closed, the volumetric flow rate of air during the Hold State is 0 L/min. As the air inlet valve opens to transition the system to the Airflow State, the pressure at the upstream pressure sensor (Pv) drops to approximately 0 mmHg, or ambient pressure levels. The pump is controlled to maintain 80 mmHg at the downstream pressure sensor (Pp) during the Airflow State. The Airflow State is operated for a 14 second duration. Following the Airflow state, the air inlet valve is closed and the system transitions to the Pressurise State where the volumetric flow rate of air rapidly decreases to 0 LPM with the pump controlled to achieve 100 mmHg at the Pv pressure sensor. The cycle continues with a further Hold State.

During the Airflow State the air volumetric flow rate achieves an equilibrium of approximately 0.111 LPM, or 111 mL/min, after approximately 3.7 seconds of the air inlet valve opening. Throughout the total 14 second duration of the Airflow State the system achieves an average airflow rate of 106 mL/min which equates to 25 mL of air being delivered through the system. The system has a volume of 27 mL. Therefore, the volume of air delivered through the system in a single 14 second Airflow cycle is approximately 75% of the internal volume of the treatment system. An increase in the air inlet valve open time from 14 seconds to 16 seconds would deliver approximately 28 mL of air through the treatment system, which would equate to approximately 100% of the internal volume of the treatment system.

With regards to the above example setup comprising a total internal volume of 16 mL, it is expected that, for the same system operation, a similar air flow rate would be achieved during the 14 second valve open duration, resulting in a total volume of approximately 25 mL of air being delivered through the system. This volume of air equates to approximately 150% of the volume of the treatment system. It is suggested that the volume of air delivered to the system should be at least 50% of the total system volume, or at least 60%, or at least 70%, or at least 80%, or at least 90%, or at least 100% of the total system volume. However, the testing shows that multiple airflow cycles are required to remove ˜99% of the fluid contained at the treatment site, as indicated by the results presented in Table 2 above. It should be noted that the inlet valve must be cycled opened and closed. If the air inlet valve remains open continuously, or is open for an excessive length of time, an annular type flow may result. Further, having the air inlet valve open continuously can result in the pump running continuously, which is undesirable for a portable system. It is suggested that the maximum volume of air delivered through the system in one valve cycle is less than 200% of the total system volume, or is less than 300% of the total system volume, or is less than 400% of the total system volume.

### Simulated Blockage Testing

A series of experiments were conducted to compare the blockage characteristics of several medium to large sized commercially available diaphragm pumps in comparison to the pump described below and with reference to FIGS. 13 to 16. The test pump device comprised an inlet 56 with an outer diameter of Ø6.8 mm and an inner diameter of Ø3 mm, and a pump outlet 57 with an outer diameter of Ø4.6 mm and inner diameter of Ø3 mm. The inlet channel 61 of the pump was connected to two inlet valves 54 in fluid connection to two separate chambers 53 with the outlet flow path comprising two outlet valves 55 in fluid connection with the outlet channel 62 and pump outlet 57. The two inlet valves 54 and two outlet valves 55 comprised duck bill valves moulded from liquid silicone rubber (LSR) with an overall height of 5 mm and an internal opening of Ø2.8 mm leading to a tapered valve closing at the apex of the valve, positioned at the furthermost distance away from the inlet opening of the valve. The overall assembly of the valve is comparable to the sectional view shown in FIG. 14.

The table below outlines the performance characteristics of the three commercially available diaphragm pumps that were tested.

TABLE 1
Commercially available diaphragm pumps

| | | | | Water | Inlet | Approx. |
|---|---|---|---|---|---|---|
| | | Rated | Rated | Free | Outer | Inlet |
| | | Volt- | Cur- | Flow | Diam- | Inner |
| Manu- | | age | rent | Rate | eter | Diameter* |
| facturer | Model | (V) | (mA) | (L/min) | (mm) | (mm) |
| Kamoer ® | KLC- | DC 12 | <220 | >1.0 | Ø6.5 | Ø5.1* |
| Fluid | A | | | | | |
| Tech | | | | | | |
| (Shanghai) | | | | | | |
| CO. LTD. | | | | | | |
| Xiamen | AJK- | DC 12 | <400 | >1.0 | Ø6.1 ± | Ø4.4* |
| AJK ™ | B2713 | | | | 0.3 | |
| Technology | | | | | | |
| CO. | | | | | | |
| LTD. | | | | | | |
| Xiamen | AJK- | DC 12 | <500 | >1.8 | Ø8 ± | Ø6.6* |
| AJK ™ | B3204 | | | | 0.3 | |
| Technology | | | | | | |
| CO. | | | | | | |
| LTD. | | | | | | |

*Assumes inlet boss wall thickness of 0.7 mm

The test media was prepared by combining and stirring 30 grams of chia seeds to 300 grams of water (300 mL) and leaving to thicken for at least 12 hours, allowing the chia seeds to soak and soften.

Chia seeds are typically small ellipsoidal shaped seeds that measure 2.15 mm×1.40 mm×0.83 mm on average when dry² and absorb significant amounts of liquid to produce a polysaccharide based gel³ that appears to form a sticky gelatine like substance that demonstrates similarities to a fibrin or fibrinogen blockage when passed through a system.

For each test a suitable tube connected the inlet of each diaphragm pump to a glass beaker full of chia seed gel with a suitable outlet tube returning the chia seed gel to the same glass beaker. The chia seed gel was pulled through the pump for several minutes where the absence of chia seeds passing from the outlet was noted as a blockage. Each of the three commercially available diaphragm pump devices blocked in under a minute of testing as shown in the table below.

TABLE 2
Blockage test results for the three commercial
diaphragm pump devices

| | | Simulated Blockage |
|---|---|---|
| Manufacturer | Model | Test Result |
| Kamoer ® Fluid Tech | KLC-A | Blocked (FAIL) |
| (Shanghai) CO. LTD. | | |
| Xiamen AJK ™ | AJK-B2713 | Blocked (FAIL) |
| Technology CO. LTD. | | |
| Xiamen AJK ™ | AJK-B3204 | Blocked (FAIL) |
| Technology CO. LTD. | | |

The test pump described herein continued to output the chia seed gel into the test beaker for several minutes during testing, indicating a PASS result.

### Animal Studies

A series of animal studies were performed to compare the effect of various valve cycle timings on clinical outcomes for seroma prevention within a unilateral ovine external abdominal oblique dead space seroma model.

The animal studies utilised an implanted wound treatment device 3 similar in shape to the one illustrated in FIG. 56. The implanted wound treatment device 3 comprised a perforated central conduit 3a approximately 260 mm long comprising a repeating row of four Ø0.5 mm±0.2 mm sized perforations spaced approximately 2 mm apart along a central conduit 3a with an approximate internal conduit area of 18 mm² (equivalent internal tube diameter of Ø4.8 mm). The implanted wound treatment device had an outer diameter of approximately 120 mm with an inner diameter of approximately 60 mm.

A removal conduit 11 approximately 1000 mm long with an internal diameter of 3/16″ (Ø4.8 mm ID) was connected to a downstream end 3c of the perforated central conduit 3a, with a supply conduit 12 approximately 1000 mm long with an internal diameter of 1/16″ (Ø1.6 mm ID) was connected to an upstream end 3b of the central conduit 3a.

Each implanted wound treatment device was connected to an externally mounted vacuum device 2 constructed to reflect the embodiment treatment system 100 represented in FIGS. 6 and 7, with the treatment algorithm as described above in relation to FIGS. 46 to 50.

The external vacuum pump device 2 connected to this implant 3 was configured to open the air inlet valve for 14 Seconds, with the closed duration time varied to assess the difference in clinical outcomes associated with varying hold lengths. Tests were carried out with a 20 second, 120 second, 240 second and 360 second valve close timing in the ‘HOLD STATE’.

The system was maintained at a vacuum pressure level of 80 mmHg during the instillation of filtered air during the AIRFLOW STATE with the system returning to a second equilibrium pressure of 100 mmHg during the PRESSURISE STATE. This cycle operated in a continuous pattern with the vacuum pressure level along the fluid removal conduit 11 capped at 150 mmHg as a safety mechanism.

Tests were carried out in five sheep, with each animal receiving a single implanted wound treatment device 3. A defect site of ˜110 cm² in area was created by excising approximately 60 grams of external abdominal oblique muscle from an undermined area above the muscle. The implant device was positioned at the lowermost ventral aspect of the defect site and was secured to the treatment site using a series of passed sutures that were tied off to affix the implant in place. The removal conduit 11 and supply conduit 12 both exiting the wound at the upper and forward most ventral-cranial aspect of the wound with the conduits held in place at the skin portal using stay sutures. Once the treatment site was closed the implant device was connected to the externally mounted vacuum pump device 2 to function as programmed.

An ultrasound assessment was performed at days 7 post-surgery to assess the size of any seroma forming at the defect site, where the volume of any seroma measured at the defect site was calculated using the formula to determine the volume of an ellipsoid.

The volume of wound exudate collected within the reservoir of the device was measured daily to determine the total amount of fluid collected over the 7 days post-surgery. All animals were euthanised at 14 days post-surgery to perform a gross assessment of the treatment site; with exception of Animal ID 5 which was euthanised 7 days post-surgery. The results from the animal study are shown in the table below.

TABLE 1
Animal study results

| | | | | Ultrasound | | |
|---|---|---|---|---|---|---|
| | | | | Seroma | Total | |
| | Valve | | Resected | Volume at 7 | Exudate | Seroma |
| | Closed | Animal | Muscle | Days Post- | Collected | Observed |
| Animal | Time | Weight | Weight | Surgery | Following 7 | Following |
| ID | (seconds) | (KG) | (grams) | (mL) | Days (mL) | Euthanasia |
| 1 | 20 | 62 | 71.0 | 0 | 369.9 | No |
| 2 | 20 | 60 | 61.8 | 0 | 280.3 | No |
| 3 | 120 | 61 | 63.5 | 0 | 188.9 | No |
| 4 | 360 | 63 | 58.9 | 156.2 | 736.2 | Yes, Large |
| 5 | 240 | 59 | 57.8 | 4.6 | 306.2 | *Yes, Minor |

*Euthanasia performed at 7-days post-surgery.

There were no signs of any seroma or wound fluid at the defect site for animal IDs 1, 2 and 3 following euthanasia at 14 days post-surgery time point, with the implanted wound treatment device 3 found to be completely integrated with the surrounding tissue.

There were moderate signs of a seroma at the defect site for Animal ID 5 which was euthanised 7 days post-surgery, with the result also consistent with the ultrasound assessment at the same time point.

The defect site of Animal ID 4 was found to have a large seroma at the 14 day post-surgery time point which was consistent with the ultrasound findings at the 7-day post-surgical timepoint with virtually zero signs of any integration of the separated tissue planes of the defect site.

The results from this animal study support the conclusion that an air inlet valve closed time of 120 seconds or less is more likely to lead to complete dead space closure and the prevention of seroma formation at the defect site with an animal.

A system according to embodiments described herein provides significant benefits, including but not limited to one or more of the following:
Improved fluid removal from the wound site, providing improved healing benefits such as reduced edema by the removal of excess exudate;
Reduced risk of blockages forming in the system;
Maintaining effective negative pressure at the wound even during addition of air to ensure effective treatment;
Removal of exudate from a lower portion of a wound where there is a height differential at the wound;
Low power consumption suited for application in portable wound treatment systems;
Application of treatment fluids to the wound while maintaining effective negative pressure at the wound to ensure effective treatment;
Provision of negative pressure to a larger portion of a treatment space to improve treatment throughout the entire treatment space;
System configurability with and without the provision of a treatment fluid supply to the wound;
Ease of providing a sterile interface between an air inlet and a wound site.

### REFERENCES

1. Malmsjö, M., Huddleston, E., & Martin, R. (2014). Biological effects of a disposable, canisterless negative pressure wound therapy system. Eplasty, 14.
2. Ixtaina, V. Y., Nolasco, S. M., & Tomas, M. C. (2008). Physical properties of chia (Salvia hispanica L.) seeds. Industrial crops and products, 28(3), 286-293.
3. Coorey, R., Tjoe, A., & Jayasena, V. (2014). Gelling properties of chia seed and flour. Journal of food science, 79 (5), E859-E866.

1. A system for treating a wound comprising:
a fluid input and a fluid output for connection to a wound treatment device located at the wound, the fluid input adapted to be fluidly connected to an upstream side of the wound treatment device and the fluid output adapted to be fluidly connected to a downstream side of the wound treatment device;
an air inlet valve upstream of the fluid output;
an actuator to drive the air inlet valve between an open position and a closed position;
a pump downstream of the fluid input;
a motor to drive the pump to provide a negative pressure to the wound treatment device; and
a controller in communication with the actuator and the motor to operate the air inlet valve and the pump; wherein the controller is configured to:
i) open the air inlet valve and operate the pump to maintain a first vacuum pressure at the wound treatment device and introduce air into the wound treatment device;
ii) close the air inlet valve and operate the pump to maintain a second vacuum pressure at the wound treatment device and remove air and fluid from the wound treatment device;
wherein the first vacuum pressure is less than or equal to the second vacuum pressure.

2. The system as claimed in claim 1, wherein the controller is configured to operate the pump to continuously maintain a negative pressure environment at the wound treatment device when the air valve is open and closed.

3. The system as claimed in claim 2, wherein the first and second vacuum pressures provide for effective negative pressure wound therapy.

4. The system as claimed in claim 1, wherein the controller is configured to repeat steps i) and ii) to cycle the air inlet valve between the open and closed positions.

5. (canceled)

6. The system as claimed in claim 1, wherein the controller is configured to operate the pump when the air inlet valve is open to maintain a substantially constant first vacuum pressure.

7. The system as claimed in claim 1, wherein the controller is configured to operate the pump with the air inlet valve open so that a flow rate of air into the system through the air inlet valve is equal to a flow rate of the pump.

8. The system as claimed in claim 1, wherein the controller is configured to operate the pump when the air inlet valve is closed to maintain a substantially constant second vacuum pressure.

9. The system as claimed in claim 1, wherein the controller is configured to:
in step (i), operate the pump with the air inlet valve open so that the system is in an equilibrium state with a zero or constant pressure differential across the treatment device.

10. The system as claimed in claim 9, wherein the controller is configured to: in step (ii), operate the pump with the air inlet valve closed so that the system is in an equilibrium state with a zero or constant pressure differential across the treatment device.

11. The system as claimed in claim 1, wherein the controller is configured to operate the air inlet valve between open and closed to introduce a flow rate of air into the system that generates a bubble flow or slug flow comprising bubbles or slugs of air entrained in fluid flow from the wound treatment device.

12. The system as claimed in claim 1, wherein the controller is configured to operate the air inlet valve between open and closed to reduce a density of fluid at the wound to lift the fluid from the wound against gravity.

13. (canceled)

14. The system as claimed in claim 1, wherein in step i) the controller is configured to open the air inlet valve for a predetermined time period.

15. The system as claimed in claim 1, wherein in step ii) the controller is configured to close the air inlet valve for a predetermined time period.

16. The system as claimed in claim 1, wherein in step i) the controller is configured to open the air inlet valve for at least 10 seconds.

17. The system as claimed in claim 1, wherein the air inlet valve is open for at least 10% of the cycle pitch, or at least 20% of the cycle pitch, or at least 30% of the cycle pitch, or at least 40% of the cycle pitch, or at least 50% of the cycle pitch.

18. The system as claimed in claim 1, wherein, in step i), the air inlet valve is open for a sufficient time period so that a volume of air delivered through the system is at least a substantial portion of a total volume of the system.

19. The system as claimed in claim 18, wherein, in step (i), the air inlet valve is open for a sufficient time period so that the volume of air delivered to the system is at least 50%, or at least 100% of the total volume of the system.

20. The system as claimed in claim 1, wherein the first vacuum pressure is about 30% to 100% of the second vacuum pressure.

21. The system as claimed in claim 1, wherein the first vacuum pressure is about 50 to 100 mmHg.

22. The system as claimed in claim 20, wherein the second vacuum pressure is about 100 to 150 mmHg.

23. The system as claimed in claim 1, wherein the first vacuum pressure is about 10 to 50 mmHg less than the second pressure.

24. The system as claimed in claim 1, wherein in step (i) the controller is configured to operate the pump to achieve a vacuum pressure threshold, wherein in step (ii) the controller is configured to operate the pump to achieve a vacuum pressure threshold.

25. (canceled)

26. The system as claimed in claim 24, wherein the system comprises:
a downstream pressure sensor located downstream of the wound treatment device and in communication with the controller, and
the controller is configured to, in step i) operate the pump to achieve the vacuum pressure threshold based on a pressure sensed by the downstream pressure sensor.

27. The system as claimed in claim 24, wherein the system comprises:
an upstream pressure sensor located upstream of the wound treatment device and in communication with the controller, and
the controller is configured to, in step ii), operate the pump to achieve the vacuum pressure threshold based on a pressure sensed by the upstream pressure sensor.

28. The system as claimed in claim 24, wherein the system comprises:
an upstream pressure sensor located upstream of the wound treatment device and in communication with the controller,
a downstream pressure sensor located downstream of the wound treatment device and in communication with the controller, and
the controller is configured to, in step i) operate the pump to achieve a first vacuum pressure threshold based on a pressure sensed by the downstream pressure sensor; and
in step ii), operate the pump to achieve a second vacuum pressure threshold based on a pressure sensed by the upstream pressure sensor.

29. The system as claimed in claim 24, wherein the first vacuum pressure threshold is less than or equal to the second vacuum pressure threshold.

30. The system as claimed in claim 28, wherein the system comprises an inlet restriction, and the upstream pressure sensor is located upstream of the inlet restriction so that the upstream pressure sensor measures ambient pressure when the air inlet valve is open.

31. The system as claimed in claim 1, wherein the system comprises an inlet restriction to present a predetermined pressure drop between ambient pressure and a vacuum pressure at the wound treatment device.

32. The system as claimed in claim 31, wherein the system comprises a filter to filter air introduced to the system, and wherein the filter is or comprises the inlet restriction.

33. The system as claimed in claim 31, wherein the pressure drop is approximately 20 to 130 mmHg.

34. The system as claimed in claim 31, wherein, when the air inlet valve is open, substantially all pressure differential between ambient pressure and a pressure downstream of the wound treatment device is at the inlet restriction.

35. The system as claimed in claim 1, wherein the system comprises a reservoir for collecting fluid removed from the wound, and wherein the reservoir is located downstream of the pump such that fluid removed from the wound passes through the pump to the reservoir.

36-37. (canceled)

38. The system as claimed in claim 1, wherein the system further comprises the wound treatment device, the wound treatment device comprising:
a porting component to be received in an external wound cavity and substantially fill a treatment space of the wound;
a cover layer to cover the wound;
a fluid supply conduit in fluid communication with the fluid outlet, the fluid supply conduit having one or more supply conduit outlets;
a fluid removal conduit in fluid communication with the fluid inlet, the fluid removal conduit having one or more removal conduit inlets;
wherein the supply and removal conduits are placed in the treatment space with the removal conduit inlet(s) and the supply conduit outlet(s) in fluid communication with the porting component and with the outlet(s) spaced from the inlet(s) so that fluid flow from the outlet(s) to the inlet(s) is through a substantial portion of the porting component and treatment space.

39. The system as claimed in claim 1, wherein the system comprises a treatment fluid inlet upstream of the fluid outlet to connect a supply of treatment fluid.

40. The system as claimed in claim 39, wherein the system is configured so that, in step i) the introduction of treatment fluid to the wound treatment device is prevented or reduced by the introduction of air to the wound treatment device by the first vacuum pressure, and in step ii), treatment fluid is drawn to the wound treatment device by the second vacuum pressure.

41. The system as claimed in claim 39, wherein the system comprises:
a treatment fluid valve between the treatment fluid inlet and the fluid outlet, and
an actuator to drive the treatment fluid inlet valve between an open position and a closed position, wherein the controller is in communication with the fluid inlet valve actuator and the controller is configured to, in a fluid supply state:
iii). open the fluid inlet valve and operate the pump to maintain a vacuum pressure at the wound treatment device and introduce treatment fluid into the wound treatment device;
iv). close the fluid inlet valve and operate the pump to maintain a vacuum pressure at the wound treatment device and remove fluid from the wound treatment device.

42. The system as claimed in claim 41, wherein the controller is configured to operate the pump to continuously maintain a negative pressure environment at the wound treatment device when the fluid inlet valve is open and closed.

43. The system as claimed in claim 41, wherein the controller is configured to, in step (iii), operate the pump to generate a third vacuum pressure at the wound treatment device, and, in step (iv), operate the pump to generate a fourth vacuum pressure at the wound treatment device, wherein the third vacuum pressure is less than or equal to the fourth vacuum pressure.

44. The system as claimed in claim 43, wherein the third vacuum pressure is equal or similar to the first vacuum pressure and the fourth vacuum pressure is equal or similar to the second vacuum pressure.

45. The system as claimed in claim 43, wherein the third and fourth vacuum pressures provide for effective negative pressure wound therapy.

46. The system as claimed in claim 41, wherein, after closing the fluid inlet valve and operating the pump to generate the vacuum pressure at the wound, the controller is configured to:
(v) flush the treatment fluid from the wound by:
(v)(a) opening the air inlet valve and operating the pump to maintain a vacuum pressure (e.g. the first vacuum pressure) at the wound treatment device and introduce air into the wound treatment device, and
(v)(b) closing the air inlet valve and operating the pump to maintain a vacuum pressure (e.g. the second vacuum pressure) at the wound treatment device and remove fluid from the wound treatment device.

47. The system as claimed in claim 46, wherein in step (v) the controller is configured to repeat steps (v)(a) and (v)(b) a predetermined number of times (for example, three times) to remove treatment fluid from the wound.

48. The system as claimed in claim 46, wherein in the fluid treatment state, the controller is configured to repeat steps (iii) to (v) a predetermined number of times.

49. The system as claimed in claim 41, wherein the controller is configured to, in step (iv), close the fluid inlet valve, wait for a predetermined time period, and operate the pump to generate the vacuum pressure at the wound treatment device and remove fluid from the wound treatment device.

50. The system as claimed in claim 41, wherein the controller is configured to activate the fluid supply state periodically.

51. The system as claimed in claim 50, wherein a time period between activating the fluid supply state is much greater than a cycle time of the air inlet valve.

52. The system as claimed in claim 41, wherein the system comprises an upstream pressure sensor and/or a downstream pressure sensor in communication with the controller, and, in step (iii), the controller is configured to operate the pump to achieve a vacuum pressure threshold based on a pressure sensed by the upstream and/or downstream pressure sensor.

53. The system as claimed in claim 41, wherein the system comprises an upstream pressure sensor and/or a downstream pressure sensor in communication with the controller, and, in step (iv), the controller is configured to operate the pump to achieve a vacuum pressure threshold based on a pressure sensed by the upstream and/or downstream pressure sensor.

54. A pump for applying negative pressure to a wound via a wound treatment device, the pump comprising:
a drive mechanism;
at least one flexible chamber, the drive mechanism configured to drive the chamber to compress and expand the chamber;
a pair of one-way valves in fluid communication with the chamber, the pair of one-way valves comprising an inlet valve for fluid flow into the chamber, and an outlet valve for fluid flow out of the chamber;
a pump inlet in fluid communication with the at least one inlet valve; and
a pump outlet in fluid communication with the at least one outlet valve;
wherein compression of the chamber causes fluid flow from the chamber, through the outlet valve and the pump outlet, and subsequent expansion of the chamber draws fluid from the pump inlet through the inlet valve and into the chamber; and
wherein the one-way inlet and outlet valves each presents a single orifice only in a fluid flow path through the pump from the pump inlet to the pump outlet via the inlet valve, chamber and outlet valve to enable the passage of fluid and tissue debris through the valves when open.

55. The pump as claimed in claim 54, wherein the single orifice has an area when the valve is open similar to or greater than a minimum area of the fluid flow path between the pump inlet to the pump outlet.

56. The pump as claimed in claim 54, wherein the single orifice has an area similar to or greater than an area of the pump inlet.

57. A pump as claimed in claim 54, wherein each inlet and outlet valve comprises a unitary flexible valve member.

58. A pump as claimed in claim 54, wherein each one-way inlet and outlet valve comprises a duck bill valve.

59. A pump as claimed in claim 54, wherein any one or more of the one-way inlet and outlet valves comprises a flapper valve, scupper valve, check valve, cross-slit valve and a dome valve.

60. The pump as claimed in claim 54, wherein the drive mechanism comprises a motor and a swash plate, rotation of the swash plate driven by the motor, the at least one chamber connected to the swash plate to compress and expand with rotation of the swash plate.

61. A pump as claimed in claim 60, wherein each chamber comprises an associated connector, the connector being attached to the chamber and to the swash plate such that it moves axially to effect compression and expansion of the respective reservoir with movement of the swash plate.

62. A pump as claimed in claim 54, comprising a plurality of flexible chambers and a plurality of pairs of inlet and outlet valves, each pair of inlet and outlet valves corresponding to a respective chamber.

63-80. (canceled)

81. A portable vacuum unit for a wound treatment system for providing negative pressure treatment to a wound, the vacuum unit comprising:
an air inlet valve;
an actuator to drive the air inlet valve between an open position and a closed position;
a pump comprising a pump inlet and a pump outlet;
a motor to drive the pump; and
a controller in communication with the actuator and the motor to operate the air inlet valve and the pump to apply negative pressure treatment to a wound, and
an interface manifold comprising:
a first fluid flow path with a first inlet and first outlet, the first inlet connected to the air inlet valve and the first outlet providing a vacuum unit fluid outlet for connection to an upstream side of the treatment device, and
a second fluid flow path with a second fluid inlet and a second fluid outlet, the second outlet connected to the pump inlet and the second inlet providing a vacuum unit fluid inlet for connecting to a downstream side of the treatment device,
an enclosure for housing the air inlet valve, actuator, pump, motor, controller, and interface manifold,
wherein the interface manifold is a separate assembly within the enclosure providing an interface between the air inlet valve and the upstream side of wound treatment device and an interface between the pump inlet and the downstream side of the wound treatment device.

82. The portable vacuum unit as claimed in claim 81, wherein the interface manifold comprises a one-way valve in the second flow path to prevent back flow from the pump inlet to the downstream side of the treatment device.

83. The portable vacuum unit as claimed in claim 81, wherein the vacuum unit has a port for connecting to a treatment fluid reservoir, and the first fluid flow path of the interface manifold has a treatment fluid inlet connected to the port to fluidly connect the treatment fluid reservoir to the treatment device.

84. The portable vacuum unit as claimed in claim 83, wherein the portable vacuum unit comprise a connection assembly comprising the manifold, the port, and a tube connecting the port to the treatment fluid inlet of the interface manifold, the connection assembly providing a sterile connection assembly between the treatment fluid reservoir and the treatment device.

85. The portable vacuum unit as claimed in claim 84, wherein the portable vacuum unit comprises a fluid inlet pinch valve configured to pinch the tube in a closed position and release the tube in an open position.

86. The portable vacuum unit as claimed in claim 81, wherein the interface manifold has a housing and the first outlet and second inlet are arranged together on the interface manifold housing for connection to a dual lumen conduit comprising a supply lumen providing air from the vacuum unit to the treatment device and a removal lumen to transfer fluid from the treatment device to the vacuum unit.

87. The portable vacuum unit as claimed in claim 81, wherein the interface manifold comprises a sterile filter in the first fluid path to filter air entering the first inlet, the interface manifold providing a sterile interface between the air inlet valve and the upstream side of wound treatment device.

88. The portable vacuum unit as claimed in claim 85, wherein the vacuum unit comprises a pressure sensor, the interface manifold providing a sterile interface between the pressure sensor and the upstream side of wound treatment device.

89. The portable vacuum unit as claimed in claim 81, wherein the manifold is releasably connected to the pump within the vacuum unit enclosure.

90. The portable vacuum unit as claimed in claim 81, wherein the interface manifold connects directly to the pump inlet.

91. The system as claimed in claim 1, wherein the controller is configured to do one or more of the following based on a pressure differential measured across the treatment device:
close the air inlet valve when the pressure differential decreases below a lower threshold;
open the air inlet valve when the pressure differential increases above an upper or maximum threshold;
stop the pump when the pressure differential increases above an upper or maximum threshold;
start the pump when the pressure differential decreases below a lower threshold.

92. The system as claimed in claim 1, wherein the controller is configured to open or close the air inlet valve and/or operate the pump based on a measured pressure differential across the treatment device.
